(19) United States
(12) Patent Application Publication
(10) Pub. No.: US 2026/0305989 A1
(43) Pub. Date: Oct. 8, 2026

(54) INTERNAL SKIN MEASUREMENT APPARATUS

(71) Applicant: National University Corporation Chiba University, Chiba-shi (JP)

(72) Inventors: Masahiro Takei, Chiba-shi (JP); Marlin Ramadhan Baidillah, Chiba-shi (JP); Isnan Nur Rifai, Chiba-shi (JP); Kiagus Aufa Ibrahim, Chiba-shi (JP)

(21) Appl. No.: 19/480,440

(22) PCT Filed: May 2, 2024
(86) PCT No.: PCT/JP2024/016863

§ 371 Date: Oct. 31, 2025

(30) Foreign Application Priority Data
May 2, 2023 (JP) 2023-076258

(51) Int. Cl. A61B 5/145; A61B 5/0531; A61B 5/1495

(52) U.S. Cl. CPC A61B 5/14546; A61B 5/0531; A61B 5/1495; A61B 2560/0223

(57) ABSTRACT

An internal skin measurement apparatus including a current/voltage injection measurement unit that has a sensor including a plurality of electrodes to be arranged on a skin at intervals each other, a concentration distribution measurement unit to measure at least one of an electrolyte concentration distribution or a blood protein concentration distribution of a specific layer inside the skin, in which the concentration distribution measurement unit includes a specific skin layer separation unit to calculate a predetermined feature including information on the specific layer, the feature being obtained based on variation quantities of a current, a potential difference, and/or a phase measured by the current/voltage injection measurement unit, and a concentration calculation unit to calculate, using the difference data, at least one of a conductivity, a permittivity, or a phase that is correlated with an electrolyte concentration and a blood protein concentration in the specific layer.

### TECHNICAL FIELD

The present invention relates to an internal skin measurement apparatus.

Priority is claimed on Japanese Patent Application No. 2023-076258, filed May 2, 2023, the content of which is incorporated herein by reference.

### BACKGROUND ART

The skin is composed of three layers of an epidermis layer having a thickness of about 0.12 to 0.2 mm, a dermis layer having a thickness of about 1.8 mm, and a subcutaneous tissue (fat layer tissue). In the medical field and the beauty field, it is required to accurately know the state of a specific layer inside the skin, such as the dermis layer. On the other hand, as the device to be used, such as a sensor, a device as simple and minimally invasive as possible, for example, a planar sensor that only needs to attached to the skin surface, is used and a technology that can visualize an electrolyte concentration distribution and a blood protein concentration distribution in a specific skin layer is required.

For example, in the beauty field, it has been reported that, due to hypothyroidism caused by stress or the like, a balance between hyaluronic acid and hyaluronidase (an enzyme that degrades hyaluronic acid) is deteriorated, and the presence of excessive hyaluronic acid in the dermis layer causes edema, and there is a demand for technology for measuring the internal state of the skin.

In addition, in the medical field, for severe burns or the like, flap surgery with vascular anastomosis is performed on transplanted skin tissue. However, due to the difficulty of the vascular anastomosis, necrosis often occurs. Therefore, to prevent necrosis as well, there is a demand for a technology for measuring the state of electrolyte concentration distribution and blood protein concentration distribution in specific skin layers such as an epidermis layer, a dermis layer, and a subcutaneous tissue.

As methods for measuring the inside of the skin, there are near-infrared cameras, optical coherence tomography (OCT), second harmonic generation light (SHG light), and photoacoustic tomography. However, these technologies are for visualizing and measuring shapes, and have not been for visualizing and measuring the electrolyte concentration distribution and the blood protein concentration distribution in specific skin layers.

Examples of technologies for visualizing the electrolyte concentration distribution and the blood protein concentration distribution in specific skin layers include the technology of Patent Document 1. Patent Document 1 discloses a method of determining a sodium ion content of a skin using bioimpedance spectroscopy, the method including applying a current to a skin of a subject at a predetermined frequency, measuring a voltage applied to the skin caused by the current, determining a resistance applied to the skin of the subject at the predetermined frequency using the measured voltage, and determining a sodium ion amount of the skin using the measured voltage. In addition, Patent Document 2 describes a bioelectric impedance measurement method in which, to simply and accurately measure information on tissue layers in the depth direction just beneath the surface of the living body (a fat layer, a muscle layer, a bone tissue layer, or the like), a biological surface electrode pad is attached to the surface of the living body, and bioelectric impedance related to information inside the living body is measured from electric signals obtained by measurement.

### CITATION LIST

### Patent Document

Patent Document 1: United States Patent Application, Publication No. 2020/0196903
Patent Document 2: Japanese Unexamined Patent Application, First Publication No. 2002-085364

### SUMMARY OF INVENTION

### Technical Problem

In the conventional bioimpedance spectroscopy technology as described above, to improve the accuracy of the image, it has been necessary to use electrode belts wound around the body or limbs as sensors, or sensors including electrodes composed of microneedles. Therefore, it has been difficult to visualize, with sufficient accuracy, the electrolyte concentration distribution and the blood protein concentration distribution in the specific skin layer by using a simple and minimally invasive sensor that does not require such structures, for example, a planar sensor that only needs to be attached to the skin surface. Therefore, to solve the above problems, the present invention proposes the following means.

### Solution to Problem

<1> An internal skin measurement apparatus according to a first aspect of the present invention includes:
a current/voltage injection measurement unit that has a sensor including a plurality of electrodes configured to be arranged on a skin at intervals from each other; and
a concentration distribution measurement unit configured to measure at least one of an electrolyte concentration distribution or a blood protein concentration distribution of a specific layer inside the skin,
in which the concentration distribution measurement unit includes a specific skin layer separation unit configured to calculate at least one feature as described below, the feature including information on the specific layer inside the skin and being obtained based on variation quantities of a current, a potential difference, and/or a phase measured by the current/voltage injection measurement unit,
(1) a feature based on a variation quantity between an impedance Z1(f) between distant electrodes through which an electric field penetrates a specific skin layer and an impedance Z2(f) between close electrodes through which the electric field does not penetrate the specific skin layer but penetrates only an upper layer of the specific skin layer,
(2) a feature based on variation quantities of a potential difference and a phase between the distant electrodes and between the close electrodes in a case where a square-wave current is applied, or a feature based on variation quantities of a current and a phase between the distant electrodes and between the close electrodes in a case where a square-wave voltage is applied, or
(3) a feature obtained by performing predetermined processing in accordance with a threshold value based on an inter-electrode distance, amplifying a potential difference or a current including information on a concentration distribution of a target component in the specific skin layer of interest, and reducing a potential difference or a current including information on the specific skin layer of no interest, and
the concentration distribution measurement unit includes a concentration calculation unit configured to calculate, using the difference data, at least one of a conductivity, a permittivity, or a phase, which is correlated with an electrolyte concentration and a blood protein concentration in the specific layer.

<2> In a second aspect of the present invention, in the internal skin measurement apparatus according to the first aspect, the concentration calculation unit may include a calibration curve comparison unit configured to calculate a concentration of an electrolyte, a blood protein, or the like based on
(a) a calibration curve of an electrolyte concentration with respect to a distribution function based on a relaxation time, which has been created in advance, and a distribution function based on the relaxation time, which is estimated from the feature, or
(b) a calibration curve of an electrolyte concentration and a power spectral density drop, which has been created in advance, and a power spectral density drop, which is estimated from the feature.

<3> In a third aspect of the present invention, in the internal skin measurement apparatus according to the second aspect, the concentration calculation unit may include the calibration curve comparison unit configured to calculate the concentration of the electrolyte, the blood protein, or the like based on the calibration curve of the electrolyte concentration with respect to the distribution function based on the relaxation time, which has been created in advance, and the distribution function based on the relaxation time, which is estimated from the feature.

<4> In a fourth aspect of the present invention, in the internal skin measurement apparatus according to the second aspect, the concentration calculation unit may include the calibration curve comparison unit configured to calculate the concentration of the electrolyte, the blood protein, or the like based on the calibration curve of the electrolyte concentration with respect to the power spectral density drop, which has been created in advance, and the power spectral density drop, which is estimated from the feature.

<5> In a fifth aspect of the present invention, in the internal skin measurement apparatus according to the first aspect, the concentration distribution measurement unit may include
a specific skin layer separation unit configured to perform the predetermined processing on the current or the potential difference measured by the current/voltage injection measurement unit in accordance with the threshold value based on the inter-electrode distance; and
an electrical property distribution calculation unit configured to calculate at least one of a conductivity distribution, a permittivity distribution, or a phase distribution based on the current or the potential difference after the predetermined processing.

<6> A measurement method of an electrolyte concentration distribution and a blood protein concentration distribution in a specific skin layer of a sixth aspect, include
calculating at least one feature as described below, the feature including information on the specific layer inside the skin and being obtained based on variation quantities of a current, a potential difference, and/or a phase measured by the current/voltage injection measurement unit that has a sensor including a plurality of electrodes configured to be arranged on a skin at intervals from each other,
(1) a feature based on a variation quantity between an impedance Z1(f) between distant electrodes through which an electric field penetrates a specific skin layer and an impedance Z2(f) between close electrodes through which the electric field does not penetrate the specific skin layer but penetrates only an upper layer of the specific skin layer,
(2) a feature based on variation quantities of a potential difference and a phase between the distant electrodes and between the close electrodes in a case where a square-wave current is applied, or a feature based on variation quantities of a current and a phase between the distant electrodes and between the close electrodes in a case where a square-wave voltage is applied, or
(3) a feature obtained by performing predetermined processing in accordance with a threshold value based on an inter-electrode distance, amplifying a potential difference or a current including information on a concentration distribution of a target component in the specific skin layer of interest, and reducing a potential difference or a current including information on the specific skin layer of no interest; and
using a result obtained by calculating, using the feature, at least one of a conductivity, a permittivity, or a phase, which is correlated with an electrolyte concentration and a blood protein concentration in the specific layer.

<7> In a seventh aspect of the present invention, in the measurement method according to the sixth aspect, the calculation using the feature may be a calculation of a concentration of an electrolyte or a blood protein based on
(a) a calibration curve of a distribution function based on the electrolyte concentration and a relaxation time, which has been created prior to the calculation of the concentration of the electrolyte or the blood protein, and a distribution function based on the relaxation time, which is estimated from the feature, or
(b) a calibration curve of an electrolyte concentration and a power spectral density drop, which has been created prior to the calculation of the concentration of the electrolyte or the blood protein, and a power spectral density drop, which is estimated from the feature.

### Advantageous Effects of Invention

According to the aspect of the present invention, it is possible to provide an internal skin measurement apparatus that can separate each layer of the specific skin layer and understand the internal state of the skin based on the measured impedance.

### BRIEF DESCRIPTION OF DRAWINGS

FIG. 1 A schematic diagram of an internal skin measurement apparatus according to the first embodiment.

FIG. 2 A schematic diagram of a current/voltage injection measurement unit of FIG. 1.

FIG. 3 A diagram showing a configuration of an electrode and a skin in a state where the electrode is in contact with the skin.

FIG. 4 An equivalent circuit of the electrode and the skin of FIG. 3.

FIG. 5 An example of a basis function gm(ln τ) in a case where the number of basis functions M is 3 (M being the maximum value of a variable m).

FIG. 6 A diagram showing a relationship between m and each element θ of a fitting parameter vector in a case where the number of the basis functions M is 17 (M being the maximum value of a variable m).

FIG. 7 An example of an estimated distribution function γ\hat{ }(ln τ) in a case where the number of the basis functions M is 3 (M being the maximum value of a variable m).

FIG. 8 An example of a distribution function and a calibration curve estimated from an impedance measured by varying the sodium chloride aqueous solution concentration c.

FIG. 9 A schematic diagram of an internal skin measurement apparatus according to the second embodiment.

FIG. 10 An example of a square-wave applied current i(t), a measured potential difference v1 between distant electrodes, a measured potential difference v2 between close electrodes, and a difference Δv between v1 and v2.

FIG. 11 An example of a calculated power spectral density (PSD) drop.

FIG. 12 An example of a calibration curve of a power spectral density drop ΔP obtained by varying the sodium chloride aqueous solution concentration c.

FIG. 13 A schematic diagram of an internal skin measurement apparatus according to the third embodiment.

FIG. 14 A schematic diagram of a current/voltage injection measurement unit of FIG. 13.

FIG. 15 A measurement pattern of a four-terminal quasi-adjacent method.

FIG. 16 A diagram for illustrating processing using a spatial voltage threshold (SVT).

FIG. 17 An example of image reconstruction showing a sodium ion concentration distribution.

FIG. 18 A schematic diagram of each specific skin layer used in the electromagnetic simulation.

FIG. 19 A diagram for illustrating a sensor arrangement, a skin structure, and an equivalent circuit of the skin, which are used in the electromagnetic simulation of Example 1.

FIG. 20 A frequency dependence of a conductivity σ and a relative permittivity ε in each specific skin layer used under the electromagnetic simulation condition of Example 1.

FIG. 21 An electromagnetic simulation result in a specific skin layer separation unit in a case where a ratio r(=φ/h_{d}) of an electrode diameter φ to a dermis layer thickness h_{d} is 1 and an inter-electrode distance d is 1 mm in Example 1.

FIG. 22 A diagram showing a change in the estimated distribution function γ\hat{ }(ln τ) when the electrode diameter φ and the inter-electrode distance d are varied in Example 1.

FIG. 23 A diagram showing an outline of an experimental apparatus of Example 2.

FIG. 24 A comparison result of a Cole-Cole plot and equivalent circuit fitting as an experimental result of Example 2.

FIG. 25 A calculation result of a specific skin layer separation unit of an experiment using a pig skin tissue in Example 2.

FIG. 26 A schematic diagram of an experimental apparatus used in Example 3.

FIG. 27 A measurement result of a measured potential difference v depending on differences in applied current frequency f_{sw} in Example 3.

FIG. 28 A measurement result of a measured voltage v depending on differences in sodium chloride aqueous solution concentration c at an applied current frequency f_{sw} of 100 kHz in Example 3.

FIG. 29 A conductance and a capacitance of the dermis layer based on time constant analysis of the sodium chloride aqueous solution concentration in Example 3.

FIG. 30 A relationship between the sodium chloride aqueous solution concentration c and a normalized PSD drop ΔP of the dermis layer of a pig skin in a case where a square-wave applied current f_{sw} of 25 kHz to 150 kHz is used in Example 3.

FIG. 31 A comparison between the electromagnetic simulation and the experimental result in a relationship between the sodium chloride aqueous solution concentration c and the normalized PSD drop ΔP at f_{sw}=100 kHz in Example 3.

FIG. 32 A schematic diagram of an internal skin measurement apparatus used in Example 4.

FIG. 33 A diagram showing a relationship between a measurement pattern m and a normalized compensation voltage <v*> in a case where a threshold value d^{Γ} is used as a parameter in Example 4.

FIG. 34 An experimental result of a reconstructed image of a sodium ion concentration distribution in a case where the threshold value d^{Γ} and the sodium chloride aqueous solution concentration are used as parameters in Example 4.

FIG. 35 A diagram showing a configuration of an electrode and a skin in a state where the electrode is in contact with the skin in individual measurement using a skip injection pattern in Example 5.

FIG. 36 A measurement pattern of a quasi-adjacent method in the individual measurement using a skip injection pattern in Example 5.

FIG. 37 A graph of distribution of relaxation time (DRT) analysis on individual measurement data using a skip injection pattern in Example 5.

FIG. 38 A graph showing a relationship between <σ> and a sodium ion concentration of a dermis layer.

FIG. 39 A comparison of spatially averaged conductivity distribution <σ*> by the electromagnetic simulation and the experiment.

FIG. 40 An evaluation result of the threshold value d^{Γ} by the electromagnetic simulation and the experiment.

### DESCRIPTION OF EMBODIMENTS

### First Embodiment

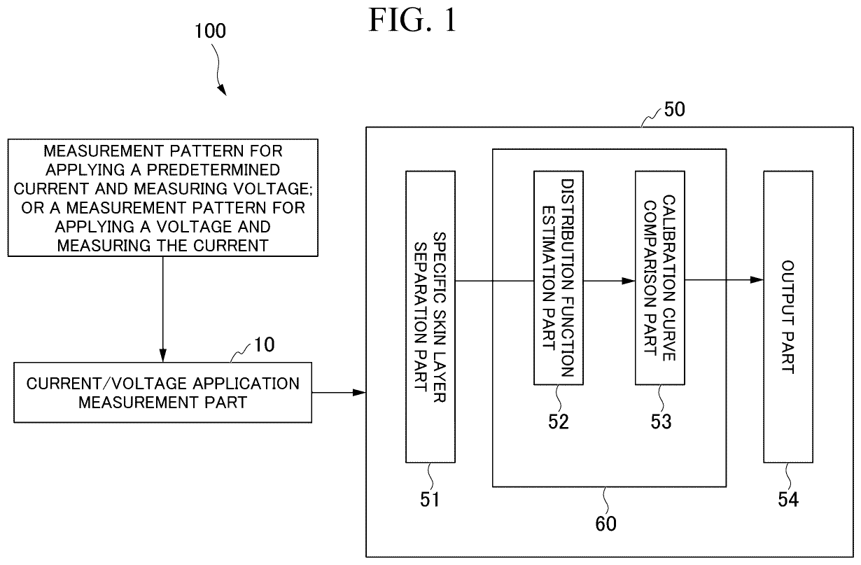

Hereinafter, an internal skin measurement apparatus according to a first embodiment of the present invention will be described with reference to the drawings. The drawing used in the following description may be enlarged for convenience in order to make the features of the present invention easy to understand, and the dimensional ratios and the like of each component may differ from the actual ones. Materials, dimensions, and the like provided in the following description are exemplary examples, and the present invention is not limited thereto, and can be appropriately modified and implemented within the range in which effects of the present invention are exhibited. As shown in FIG. 1, an internal skin measurement apparatus 100 includes a current/voltage injection measurement unit 10 and a concentration distribution measurement unit 50. The concentration distribution measurement unit 50 includes a specific skin layer separation unit 51, a distribution function estimation unit 52, a calibration curve comparison unit 53, and an output unit 54.

The concentration distribution measurement unit 50 of the internal skin measurement apparatus 100 is provided with, for example, a Central Processing Unit (CPU), a Read Only Memory (ROM), a Random Access Memory (RAM), and a Hard Disk Drive (HDD)/Solid State Drive (SSD). The specific skin layer separation unit 51, the distribution function estimation unit 52, the calibration curve comparison unit 53, and the output unit 54 are realized by executing a predetermined program in the CPU. The program may be acquired via a recording medium or may be acquired via a network. In addition, a dedicated hardware configuration may be used to realize the configuration of the internal skin measurement apparatus 100. It is noted that the concentration distribution measurement units 50A and 50B described below may have the same configuration as the concentration distribution measurement unit 50. Hereinafter, each unit will be described.

### (Current/Voltage Injection Measurement Unit)

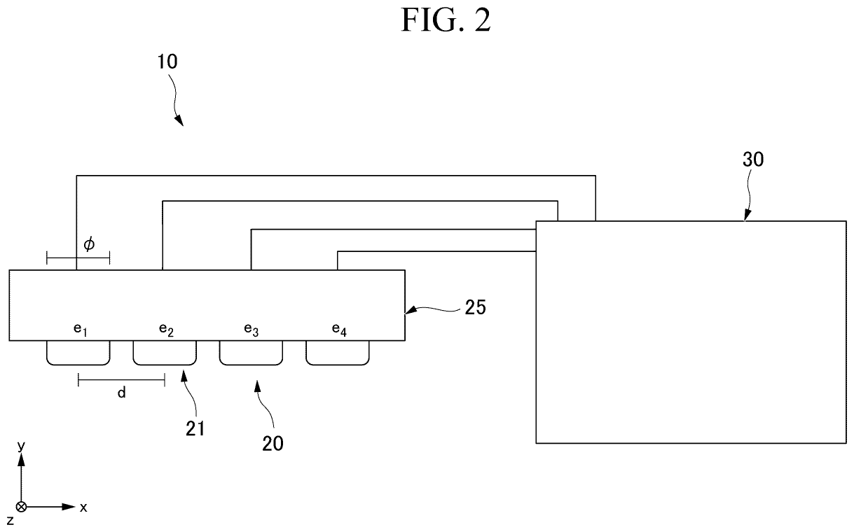

A description will be given of the current/voltage injection measurement unit 10 using FIG. 2. As shown in FIG. 2, the current/voltage injection measurement unit 10 is provided with a sensor 20 and a control unit 30. The sensor 20 is provided with a plurality of electrodes 21 (as an example, the number Q of the electrodes=4 in the diagram) configured to be arranged on the skin at intervals from each other and a support body 25 that holds the electrodes 21. The current/voltage injection measurement unit 10 has the sensor 20, applies a predetermined current or potential difference between the electrodes 21 in a state where the electrodes 21 of the sensor 20 is in contact with the skin of the person to be measured, and measures the potential difference or current. Here, the potential difference represents the difference in voltage between two electrodes. In a case of applying a current, the potential difference is measured based on a predetermined current application/voltage measurement pattern (a pattern in which two electrodes are sequentially selected from a large number of electrodes, a current is applied, and the potential difference is sequentially measured). At this time, it is desirable to also measure the phase (time lag between the applied current and the measured potential difference). In a case of applying a potential difference, the current is measured based on a predetermined voltage application/current measurement pattern (a pattern in which two electrodes are sequentially selected from a large number of electrodes, a potential difference is applied, and the current is sequentially measured). At this time, it is preferable to also measure the phase (time lag between the applied potential difference and the measured current). Below, a description will be given centering on a case in which a current is applied and detailed description of a case in which a potential difference is applied may be omitted. Here, the number Q of the electrodes 21 is, for example, 3 or more. The number Q of the electrodes 21 is preferably 8 or more. The number Q of the electrodes 21 is preferably 32 or less. The number Q of the electrodes 21 may be 16 or less. To improve the precision of the concentration distribution, it is preferable to have a large number of electrodes. The arrangement positions of the electrodes 21 are not particularly limited. For example, the electrodes may be arranged in a straight line, or the electrodes may be arranged two-dimensionally or three-dimensionally.

The electrodes 21 are electrically connected to the control unit 30. The materials and shapes of the electrodes 21 are not particularly limited as long as it is possible to apply a current or potential difference to the skin of the person to be measured. Examples of the material of the electrodes 21 include metals such as Au, Ag, Cu, and stainless steel, conductive polymers, fibers having surfaces coated with metal, fibers having surfaces coated with conductive polymers, and the like. To bring the electrodes 21 into contact with the skin, the shape of the electrodes 21 in a plane orthogonal to the current application direction is not particularly limited, but may be, for example, a circular or polygonal shape. The electrodes 21 are preferably non-invasive electrodes.

The arrangement of the electrodes 21 may be determined using electromagnetic simulation based on the size of the electrodes 21 and the distance between the electrodes 21, using a skin model in the specific skin layer consisting of the stratum corneum layer, the epidermis layer, and the dermis layer. For example, in a case where the shape of the electrode in the plane perpendicular to the current application direction is a circular shape, the diameter is used, but for example, in a case where the shape of the electrode 21 is a quadrangular shape or a polygonal shape, an equivalent circle diameter may be used. The size of the electrodes 21 and the distance between the electrodes 21 affect the identification of different specific skin layers of the skin consisting of the stratum corneum layer, the epidermis layer (excluding the stratum corneum layer), the dermis layer, and the fat layer. By optimizing the diameter of the electrode and the distance between the electrodes, the current density of only the specific skin layer of interest including a significant distribution of the electrolyte concentration and the blood protein concentration is affected.

The size (equivalent circle diameter) of the electrodes 21 is, for example, 0.2 mm to 2.0 mm. More preferably, the size is 0.4 mm to 1.2 mm. In a case where the size of the electrodes 21 is 0.2 mm to 2.0 mm, the electrolyte concentration distribution and the blood protein concentration distribution (for example, a sodium ion concentration distribution) in the specific skin layer can be more accurately visualized and measured. Here, the concentration distribution is used as a term representing a one-dimensional space-time, a two-dimensional space-time, a three-dimensional space-time, a four-dimensional space-time (for example, a three-dimensional space and time), and the concentration and the concentration distribution are not particularly distinguished from each other in terms of terminology.

The distance between the electrodes 21 is, for example, 0.5 mm to 3.0 mm. More preferably, the distance is 1.8 mm to 2.6 mm. In a case where the distance between the electrodes 21 is 0.5 mm to 3.0 mm, the electrolyte concentration distribution and the blood protein concentration distribution (for example, a sodium ion concentration distribution or the like) in the specific skin layer can be more accurately visualized and measured. The distance between the electrodes 21 is a center-to-center distance of the electrodes 21. The center of the electrode 21 may be, for example, a center of a minimum enclosing circle including the electrode 21 in a plane perpendicular to the current application direction.

An electric connection method between the electrode 21 and the control unit 30 is not particularly limited. As the connection method, for example, the electrode 21 and the control unit 30 may be connected by a lead wire.

The support body 25 is not particularly limited as long as the electrodes 21 can be held. It is preferable that the support body 25 can be deformed along a surface shape of the skin of the person to be measured. By deforming along the surface shape of the skin, the adhesiveness between the electrodes 21 and the person to be measured is improved, and more accurately apply a current or potential difference and measure the potential difference or current. As the material of the support body 25, for example, a dielectric such as an elastomer is preferable. The support body 25 is, for example, a printed board. The shape of the support body 25 is not particularly limited and examples thereof include a sheet shape, a plate shape, and the like.

### (Control Unit)

The control unit 30 is provided with, for example, a multiplexer for switching between a current applying electrode that applies a current (or a voltage applying electrode that applies a potential difference) and a voltage measuring electrode that measures a potential difference (or a current measuring electrode that measures a current), an impedance analyzer that performs voltage measurement (or current measurement) and phase measurement, and the like. An impedance analyzer is a component that measures impedance, that is, the ratio of a measured potential difference (applied potential difference) to an applied current (measured current) and the phase thereof by varying the application frequency and amplitude. For example, the control unit 30 executes a predetermined program in the CPU and controls the multiplexer and the impedance analyzer to perform impedance measurement (measurement of the ratio of the potential difference to the current and the phase thereof). The impedance measurement may be performed by controlling the control unit 30 only within the current/voltage injection measurement unit 10, or the impedance measurement may be performed by controlling the control unit 30 according to a program executed by the concentration distribution measurement unit 50. The results of the impedance measurement are sent to the concentration distribution measurement unit 50. The method for transmitting information to the concentration distribution measurement unit 50 is not particularly limited. Information may be sent from the control unit 30 to the concentration distribution measurement unit 50 through a wire, or may be sent to the concentration distribution measurement unit 50 using another transmission method.

The control unit 30 applies a current between the electrodes 21 and measures a potential difference based on a predetermined current application/voltage measurement pattern (a pattern of applying a current between which electrodes and measuring a potential difference between which electrodes). Alternatively, the control unit 30 applies a potential difference between the electrodes 21 and measures a current based on a predetermined voltage application/current measurement pattern. The control unit 30 measures the current and the phase or the potential difference and the phase using two or more electrode pairs in which the distances between the electrodes in the sensor 20 are different. In a case of applying the current and similarly in a case of applying the potential difference, the electrodes 21 between which the current (potential difference) is applied and the electrodes between which the potential difference (current) is measured are not particularly limited. For the number M of the current application/voltage measurement patterns, the current value to be applied and application frequency thereof, which differ in each current application/voltage measurement pattern, are preferably alternating current of, for example, 1.0 mA or less and from the 1 Hz band to about the 1,000 MHz band, in view of the influence on the living body and the simplicity of the apparatus.

The concentration distribution measurement unit 50 performs the difference processing on the potential difference or the current measured by the current/voltage injection measurement unit 10 to measure (visually measure) at least one of the electrolyte concentration distribution or the blood protein concentration distribution of the specific layer inside the skin. Examples of the electrolyte include a sodium ion, a potassium ion, a magnesium ion, a calcium ion, a chloride ion, and the like. Examples of the blood protein include albumin, α globulin, β globulin, γ globulin, fibrinogen, and the like. The concentration distribution measurement unit 50 includes the specific skin layer separation unit 51, a concentration calculation unit 60, and the output unit 54. The concentration calculation unit 60 includes the distribution function estimation unit 52 and the calibration curve comparison unit 53. Hereinafter, each unit will be described.

### (Specific Skin Layer Separation Unit)

The specific skin layer separation unit 51 calculates the difference data by performing the difference processing on the potential difference and the phase or the current and the phase measured by the current/voltage injection measurement unit 10. Specifically, the specific skin layer separation unit 51 calculates a target impedance that is the difference data, which is the impedance in the specific skin layer, by performing the difference processing on the two measured impedances. The difference data is sent to the distribution function estimation unit 52. Here, the specific skin layer is the stratum corneum layer, the epidermis layer, the dermis layer, the fat tissue layer, or the like. The two impedance measurement values are impedances between different electrode 21 distances. Specifically, one of the impedances to be measured is an impedance measured such that the electric field center line between the electrodes 21 passes through a specific skin layer to be targeted (target specific skin layer), and the other impedance to be measured is an impedance measured such that the electric field center line between the electrodes 21 passes through a specific skin layer other than the target specific skin layer. The distance between the electrodes 21 can be appropriately set according to the target specific skin layer.

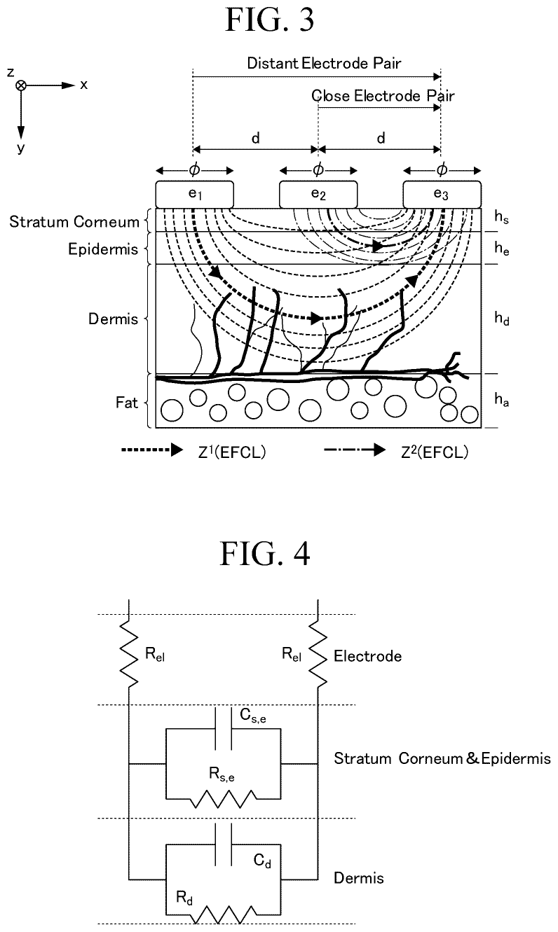

The effect of the difference processing of the specific skin layer separation unit 51 will be described below. FIG. 3 is a diagram showing a configuration of an electrode and a skin in a state where the electrode is in contact with the skin. FIG. 4 is an equivalent circuit of FIG. 3 created by assuming a six-parameter equivalent circuit in which the contact resistance of the electrode 21 and the specific skin layer (stratum corneum layer, epidermis layer (excluding the stratum corneum), dermis layer, and fat layer) are modeled as one pair of RCs. In FIG. 3, the electric field center line (EFCL) between the electrodes 21 is illustratively depicted in a non-linear manner. Here, a capacity component of the stratum corneum layer and the epidermis layer is denoted by C_{s,e}, a resistance component of the stratum corneum layer and the epidermis layer is denoted by R_{s,e}, a capacity component of the dermis layer is denoted by C_{d}, and a resistance component of the dermis layer is denoted by R_{d}. The applied current penetrates the specific skin layer under the influence of the size of the electric equivalent circuit of FIG. 4.

Since the EFCL easily passes through the specific skin layer of interest by the electrode diameter φ and the optimal inter-electrode distance d, more detailed electrical information of the specific skin layer of interest can be obtained. As the electrical information of the specific skin layer, a current or a voltage between the electrodes 21 of the sensor 10 is applied, the voltage or the current is measured, and the impedance can obtained from these values. The thickness of each specific layer is represented by, for example, hs for the stratum corneum layer, he for the epidermis layer (excluding the stratum corneum layer), hd for the dermis layer, and ha for the fat layer. The rightmost electrode of the electrodes 1 to 3 (e₁, e₂, and e₃) is set as an earth electrode. A constant current of an alternating current frequency f is applied to the leftmost electrode and the central electrode, and a potential difference between the leftmost electrode and the earth electrode (between distant electrodes) and a potential difference between the central electrode and the earth electrode (between close electrodes) are measured. For example, in a case where the dermis layer is focused on among the specific skin layers, the target impedance ΔZ(f) including the information on the dermis layer is a difference between an impedance Z¹(f) between distant electrodes through which the EFCL penetrates the dermis layer and an impedance Z²(f) between close electrodes through which the EFCL penetrates only the stratum corneum layer and the epidermis layer. Specifically, it is represented by Expression (1). Since the ΔZ(f) eliminates parasitic components between Z¹(f) and Z²(f), the impedance of the specific skin layer can be accurately extracted. It is noted that (f) represents a function of the application frequency.

$$Δ Z ( f ) = {Z}^{1} ( f ) - {Z}^{2} ( f ) \qquad ( 1 )$$

The concentration calculation unit 60 calculates at least one of the conductivity, the permittivity, or the phase that is correlated with the electrolyte concentration and the blood protein concentration in the specific layer, using the target impedance ΔZ(f) that is the difference data. The concentration calculation unit 60 may include a distribution function estimation unit 52 that estimates a distribution function γ(ln τ) from the target impedance, and a calibration curve comparison unit that calculates at least one of the electrolyte concentration or the blood protein concentration from an estimated distribution function γ\hat{ }(ln τ) estimated by the distribution function estimation unit 52.

### (Distribution Function Estimation Unit)

The distribution function estimation unit 52 estimates the distribution function γ(ln τ) from the target impedance ΔZ(f) that is the difference data. The target impedance ΔZ(f) may be directly analyzed to estimate a specific application frequency f (or a relaxation time t that is the inverse number of f) that strongly reacts to the electrolyte concentration and the blood protein concentration in the specific layer, but the specific application frequency f (or the relaxation time τ that is the inverse number of f) that strongly reacts to the electrolyte concentration and the blood protein concentration in the specific layer can be easily obtained by estimating using the distribution function γ(ln τ). Hereinafter, a method of estimating the distribution function of the distribution function estimation unit 52 will be described. The estimated distribution function is sent to the calibration curve comparison unit 53.

An impedance Z(f) (the superscript is omitted, but Z(f) corresponds to Z¹(f) or Z²(f) of Expression (1)) measured on a skin surface in a case where a constant current of the application frequency f is applied is represented by Expression (2) using a relaxation time τ in the specific skin layer, a resistance R_{∞} at an infinite frequency, and a distribution function γ(ln τ). In Expression (2), j is an imaginary unit, and ln represents a logarithm with a base e. From Expression (2), the problem of obtaining the distribution function γ(ln τ) with Z(f) being known is referred to as a mathematically ill-posed inverse problem, and many algorithms have been developed, and a known algorithm can be used. In the present embodiment, a method such as regularized least squares, algebraic reconstruction, or Bayesian estimation can be used. It is noted that the impedance used in the description of the expression above and the following expressions may be an impedance corresponding to Z¹(f) or Z²(f) of Expression (1), or may be the target impedance ΔZ(f). That is, the distribution function γ(ln τ) may be estimated by solving the ill-posed inverse problem from ΔZ(f), or by estimating the distribution functions γ¹ (ln τ) and γ²(Int) corresponding to Z¹(f) and Z²(f) by solving the ill-posed inverse problem from Z¹(f) and Z²(f), and subtracting them from each other.

The distribution function γ(ln τ) is represented by Expression (3) using M pieces of known basis functions gm(ln τ) (1≤m≤M) and M pieces of unknown fitting parameters θm (1≤m≤M). The fitting parameter θm is a parameter that adjusts an amplitude of the m-th basis function gm and has M pieces of elements. M representing the number of the fitting parameters and the number of the basis functions is not particularly limited, but is, for example, about 10 to 100. Estimating the distribution function γ(ln τ) described above means obtaining the unknown fitting parameter θm by solving the ill-posed inverse problem.

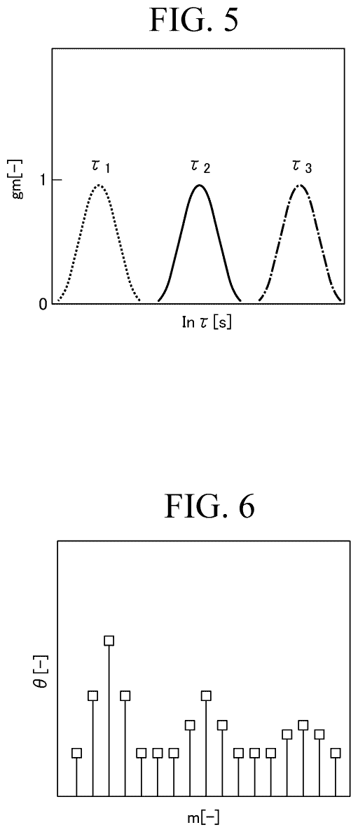

The basis function gm(ln τ) can be represented by, for example, a radial basis function such as a Gaussian basis function in which the relaxation time τ is a function, such as Expression (4). FIG. 5 is an example of the basis function gm(ln τ) in a case where the number M of the basis functions is 3 (M is the maximum value of a variable m). Here, τ is a variable, but τm (m=1, 2, 3) is a known set value, ln τm indicates a center value of the m-th Gaussian basis function, and σ² indicates a variance value.

$$Z ( f ) = {R}_{∞} + {\int }_{0}^{∞} \frac{γ ( ln τ )}{1 + j 2 π f τ} d ( ln τ ) \qquad ( 2 )$$

$$γ ( ln τ ) = {\sum }_{m = 1}^{M} {θ}_{m} {g}_{m} ( ln τ ) \qquad ( 3 )$$

$${g}_{m} ( ln τ ) = exp ( - \frac{{( \vert ln τ - ln {τ}_{m} \vert )}^{2}}{2 {σ}^{2}} ) \qquad ( 4 )$$

In a case where Expression (4) is substituted into Expression (3) and the expression is further substituted into Expression (2), the impedance Z(f) of Expression (2) is represented by Expression (6) from the unknown fitting parameter θm and the known basis function gm(ln τ). In a case where the impedance is decomposed into a real component Z^{re} and an imaginary component Z^{im}, the real component Z^{re} and the imaginary component Z^{im} are represented by Expressions (7) and (8). In practice, in a case where the unknown fitting parameter θm is obtained by solving the ill-posed inverse problem, as represented by Expression (5), a fitting parameter vector θ represented by a column vector having M pieces of elements may be used, where T in Expression (5) represents a transpose.

$$θ = {[ {θ}_{1} … {θ}_{m} … {θ}_{M} ]}^{T} \qquad ( 5 )$$

$$Z ( f ) = {R}_{∞} + {\sum }_{m = 1}^{M} {θ}_{m} {\int }_{0}^{∞} \frac{{g}_{m} ( ln τ )}{1 + j 2 π f τ} d ( ln τ ) \qquad ( 6 )$$

$${Z}^{re} = {R}_{∞} + {\sum }_{m = 1}^{M} {θ}_{m} {\underbrace{[ {\int }_{0}^{∞} \frac{{g}_{m} ( ln τ )}{1 + {( 2 π f τ )}^{2}} d ( ln τ ) ]}}_{{A}^{re}} \qquad ( 7 )$$

$${Z}^{im} = - {\sum }_{m = 1}^{M} {θ}_{m} {\underbrace{[ {\int }_{0}^{∞} \frac{2 π f τ {g}_{m} ( ln τ )}{1 + {( 2 π f τ )}^{2}} d ( ln τ ) ]}}_{{A}^{im}} \qquad ( 8 )$$

A method of obtaining the fitting parameter vector obtained by solving the ill-posed inverse problem, that is, the optimal fitting parameter vector θ* will be described. Here, since the application frequency f is redefined as the number of application frequencies and f (=1, . . . , F) pieces of application frequencies are used, the real component Z^{re} and the imaginary component Z^{im} can be represented by a column vector having F pieces of elements, a real component vector of the impedance is represented by Expression (9) and an imaginary component vector thereof is represented by Expression (10). Furthermore, in a case where A^{re} is set in the integral of the real component Z^{re} and A^{im} is set in the integral of the imaginary component Z^{im}, the real component can be represented by Expression (11) and the imaginary component can be represented by Expression (12) in an F-row and M-column matrix for corresponding to the M pieces of basis functions.

As a method of obtaining the optimal fitting parameter vector θ*, for example, ridge regression (linear regression with L2 regularization) can be used. In a case where the ridge regression is used, a cost function L(θ)=error term (loss function)+regularization term (penalty term) as a function of the fitting parameter vector θ is represented by Expression (13). Here, λ is a regularization parameter (hyperparameter) (λ>0). In Expression (13), ∥ and a subscript 2 are an L2 norm, a superscript 2 is a square, and 1∈R^{F} is a column vector having 1 as an element. The optimal fitting parameter vector θ* can be obtained by Expression (14) using L(θ). In Expression (14), argmin means obtaining the fitting parameter vector θ that minimizes L(θ). For example, in a case of M=17, θ* can be obtained as shown in FIG. 6.

$${Z}^{re} ∈ {ℝ}^{F} \qquad ( 9 )$$

$${Z}^{im} ∈ {ℝ}^{F} \qquad ( 10 )$$

$${A}^{re} ∈ {ℝ}^{F \times M} \qquad ( 11 )$$

$${A}^{im} ∈ {ℝ}^{F \times M} \qquad ( 12 )$$

$$L ( θ ) = {‖ ( ( {R}_{∞} 1 ) + {A}^{re} θ ) - {Z}^{re} ‖}_{2}^{2} + {‖ {A}^{im} θ - {Z}^{im} ‖}_{2}^{2} + λ {‖ θ ‖}_{2}^{2} \qquad ( 13 )$$

$${θ}^{*} = arg {min}_{θ : θ \geq 0} L ( θ ) \qquad ( 14 )$$

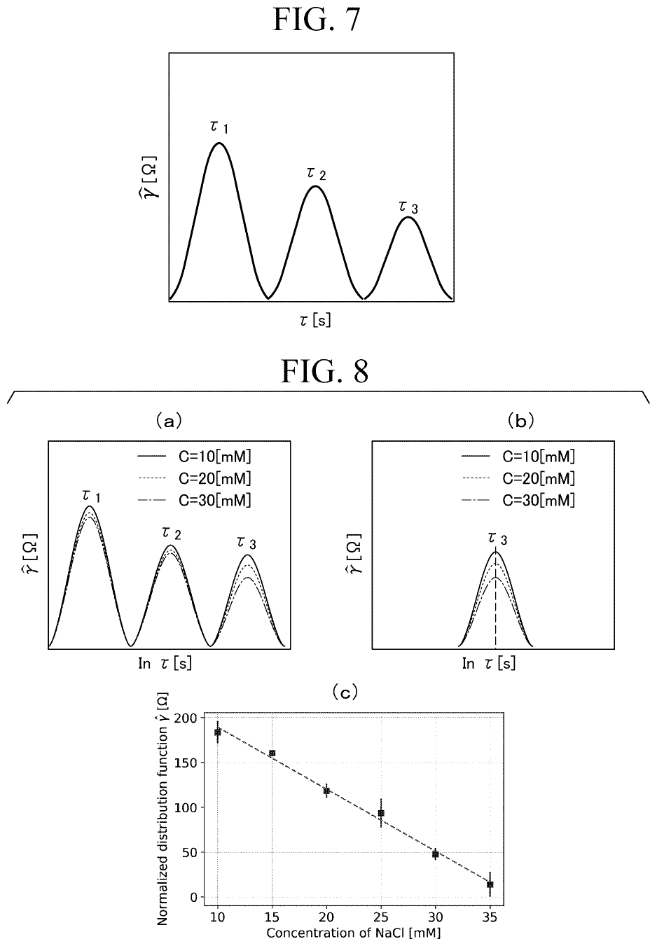

After obtaining θ*, the estimated distribution function γ\hat{ }(ln τ) obtained by substituting θ* into Expression (3) is obtained. The \hat{ } on γ indicates the estimated distribution function obtained by the optimal fitting parameter vector θ*. FIG. 7 is an example of the estimated distribution function γ\hat{ }(ln τ), and shows a series network of parallel RC circuits, each having a maximal value at a center of the corresponding relaxation time τ and each defined by a relaxation time constant τᵢ=RᵢCᵢ. That is, the relaxation time τ of the horizontal axis of the three peaks of the estimated distribution function γ\hat{ }(ln τ) corresponds to the electrolyte concentration and the blood protein concentration in each specific skin layer, and for example, the third peak strongly corresponds to the electrolyte concentration in the dermis layer, and the first peak strongly corresponds to the blood protein concentration in the dermis layer. As described above, which layer in the specific skin layer is to be visualized and measured depends on the diameter φ of the electrode 21 and the optimal inter-electrode distance d, and which type of electrolyte concentration and blood protein concentration is to be visualized and measured depends on the relaxation time τ of the peak position.

### (Calibration Curve Comparison Unit)

The calibration curve comparison unit 53 calculates at least one of the electrolyte concentration or the blood protein concentration from the estimated distribution function γ\hat{ }(ln τ) estimated by the distribution function estimation unit 52. Specifically, a content of a component to be targeted and the estimated distribution function γ\hat{ }(ln τ) corresponding to the content of the component are acquired in advance, and a calibration curve is created. The concentration of the target component (electrolyte, blood protein, or the like) is calculated from the created calibration curve and the estimated distribution function γ\hat{ }(ln τ) estimated by the distribution function estimation unit 52. FIG. 8A is an example of a distribution function estimated from an impedance measured by injecting a solution in which a known sodium chloride aqueous solution concentration c is varied into a dermis layer simulating a living body, with an electrode arrangement in which the dermis layer has a high sensitivity. FIG. 8B is a result of enlarging the rightmost peak that has changed. In this example, it can be seen that, in a case where the sodium chloride aqueous solution concentration in the dermis layer (third peak) is increased to 10 mM to 30 mM (theoretically, the sodium ion is proportionally increased), the intensity of the peak is reduced. FIG. 8C is a result of creating a calibration curve from a peak top value of the estimated distribution function γ\hat{ } of FIG. 8B and a known sodium chloride aqueous solution concentration. As described above, by creating the calibration curve in advance, the electrolyte concentration and the blood protein concentration of the specific skin layer can be detected from the estimated distribution function γ\hat{ }, and the concentration distribution of the component to be targeted can be obtained. Here, since the electrolyte concentration and the blood protein concentration in the specific skin layer to be obtained and the known solution concentration are different in a case where the degree of ionization or the degree of dissociation is not 1, the calibration curve may be corrected using the degree of ionization, the degree of dissociation, or the like. In this example, the dermis layer in the specific skin layer is taken as an example, but the calibration curve can be created in the same manner in other specific skin layers.

### (Output Unit)

The output unit 54 outputs at least one of the electrolyte concentration or the blood protein concentration sent from the calibration curve comparison unit 53. The output destination may be a display unit such as a liquid crystal display or a storage device such as an HDD.

The internal skin measurement apparatus 100 according to the first embodiment has been described above. In the above description, the specific skin layer separation unit 51 calculates the difference data by performing the difference processing on the potential difference and the phase or the current and the phase measured by the current/voltage injection measurement unit 10, and the distribution function estimation unit 52 estimates the distribution function γ(ln τ) from the difference data, but the present invention is not limited thereto. The distribution function estimation unit 52 may estimate a plurality of distribution functions γ(ln τ) based on the potential difference or the current measured, and the specific skin layer separation unit 51 may perform the difference processing on the plurality of estimated distribution functions (for example, two distribution functions) to obtain a difference estimation function.

### Second Embodiment

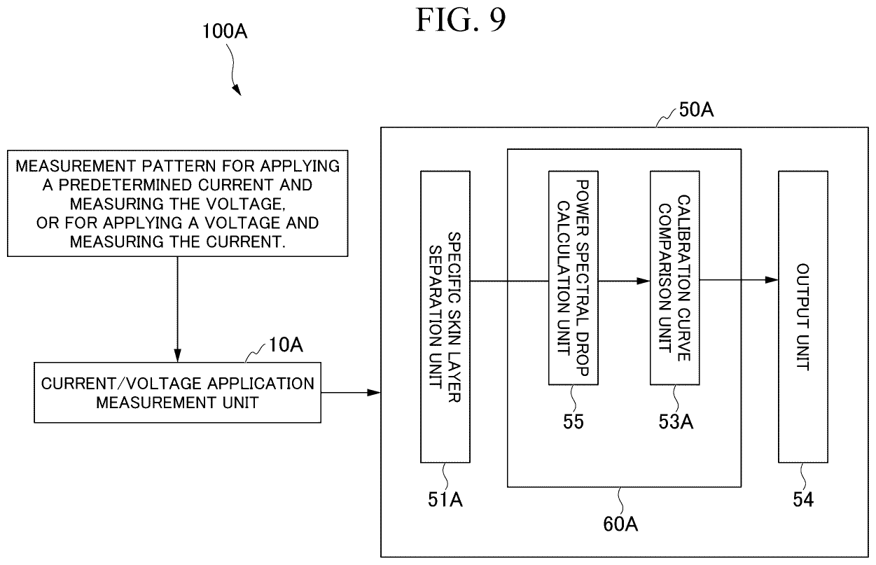

Next, an internal skin measurement apparatus 100A according to a second embodiment of the present invention will be described with reference to FIG. 9.

In addition, in the second embodiment, the same portions as the components in the first embodiment described above will be designated by the same reference signs, and a description thereof will be omitted, and only different points will be described.

As shown in FIG. 9, an internal skin measurement apparatus 100A includes a current/voltage injection measurement unit 10A and a concentration distribution measurement unit 50. A concentration distribution measurement unit 50A includes a specific skin layer separation unit 51A, a concentration calculation unit 60A, and the output unit 54.

### (Current/Voltage Injection Measurement Unit 10A)

The current/voltage injection measurement unit 10A of the second embodiment has the same configuration as the current/voltage injection measurement unit 10 of the first embodiment.

In the second embodiment, the control unit 30 applies a square-wave current or a square-wave voltage to the electrode in contact with the skin, and measures the potential difference or the current. Here, a case will be described as an example in which the rightmost electrode of the electrodes 1 to 3 (e₁, e₂, and e₃) is set as the earth electrode as in the first embodiment, a constant current of the alternating current frequency f is applied to the leftmost electrode and the central electrode, and the voltage between the leftmost electrode and the earth electrode (between distant electrodes, inter-electrode distance 2d) and the voltage between the central electrode and the earth electrode (between close electrodes, inter-electrode distance 1d) are measured.

The square-wave applied current i(t) may directly generate a square-wave or may generate by using a Fourier series, and it can be represented by, for example, Expression (15). Here, f_{sw} in Expression (15) indicates a frequency of the square-wave applied current to be generated, (2m−1) is a harmonic order, m is a natural number, M is the maximum number of the harmonics, and in a case where M is infinite, a perfect square-wave is generated. From Expression (15), the spectrum of the square-wave applied current includes an odd-order harmonic signal 2π(2m−1)f of a first order (m=1), a third order (m=2), and (m=3). An advantage of using the square-wave is that the measurement is efficient, and the reason for this is that one square-wave includes a sine wave and a cosine wave having many frequencies. That is, the reason for this is that the square-wave can be generated by using the Fourier series (sine wave and cosine wave having many frequencies). The specific skin layer has different frequency response characteristics of each specific skin layer. For example, the epidermis layer (excluding the stratum corneum layer) can be observed at 0 Hz to 10 Hz, the dermis layer can be observed at 10 Hz to 1 kHz, and the fat layer tissue can be observed at 100 Hz to 10 kHz. Since the square-wave includes various frequency components, it is easy to understand the state of each specific skin layer.

The concentration distribution measurement unit 50A performs the difference processing on the potential difference and the phase or the current and the phase measured by the current/voltage injection measurement unit 10A to measure (visually measure) at least one of the electrolyte concentration distribution or the blood protein concentration distribution of the specific layer inside the skin. The concentration distribution measurement unit 50A includes the specific skin layer separation unit 51A, a power spectral drop calculation unit 55, a calibration curve comparison unit 53A, and the output unit 54. Hereinafter, each unit will be described.

### (Specific Skin Layer Separation Unit)

The specific skin layer separation unit 51A calculates the difference data by performing the difference processing on the potential difference and the phase or the current and the phase measured by the current/voltage injection measurement unit 10A. In the second embodiment, a case where the square-wave current is applied and the potential difference and the phase are measured will be described. Here, it is assumed that v2 between close electrodes (inter-electrode distance 1d) is mainly affected by the stratum corneum layer admittance Ys and the epidermis layer admittance Ye, and v1 between distant electrodes (inter-electrode distance 2d) is mainly affected by the stratum corneum layer admittance Ys, the epidermis layer admittance Ye, and the dermis layer admittance Yd.

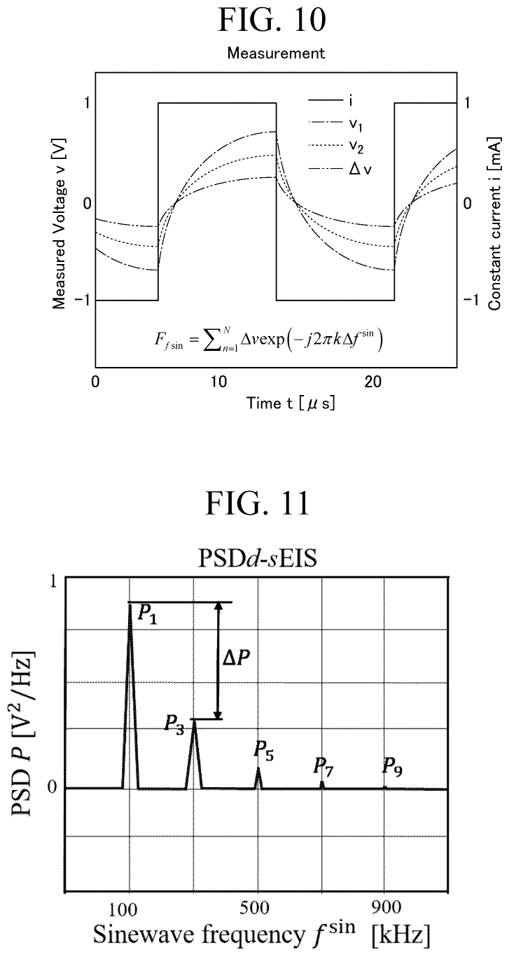

The specific skin layer separation unit 51A calculates a voltage drop Δv, which is a difference between v1 and v2, as the difference data. FIG. 10 shows an example of the square-wave applied current i(t), the measured voltage v1 between distant electrodes, the measured voltage v2 between close electrodes, and the difference Δv between v1 and v2 in the square-wave applied current and the potential difference measurement. A horizontal axis of FIG. 10 is time (μs), and a vertical axis is a potential difference (V). The calculated difference data, that is, the voltage drop ΔV is sent to the concentration calculation unit 60A.

### (Concentration Calculation Unit)

The concentration calculation unit 60A calculates at least one of the conductivity, the permittivity, or the phase that is correlated with the electrolyte concentration and the blood protein concentration in the specific layer, using the difference data. The concentration calculation unit 60A includes a power spectral drop calculation unit 55 that calculates a power spectral drop ΔP from the difference data, and a calibration curve comparison unit 53A that calculates at least one of the electrolyte concentration or the blood protein concentration from the power spectral drop ΔP calculated by the power spectral drop calculation unit 55.

### (Power Spectral Drop Calculation Unit 55)

The power spectral drop calculation unit 55 calculates the power spectral drop ΔP from the difference data.

In a case where the Fourier transform is performed on a difference Δv(t) between the measured voltage v1(t) between distant electrodes and the measured voltage v2(t) between close electrodes, Expression (16) is obtained. The power spectral density (PSD) at the harmonic order is Expression (17). Here, N is the number of voltage samples, and PSD is a function of the harmonic order (2m−1).

$${I}_{{f}^{sw} , δ} ( t ) = \frac{4}{π} {\sum }_{m = 1}^{M} \frac{1}{δ ( 2 m - 1 )} sin ( 2 π ( 2 m - 1 ) {f}_{sw} δ t ) \qquad ( 15 )$$

$${ℱ}_{{f}^{sin}} ( Δ v ( t ) ) = {\int }_{- ∞}^{∞} Δ v ( t ) exp ( - j 2 π {f}^{sin} ) dt \qquad ( 16 )$$

$${P}_{{f}^{sin}} = \frac{2}{N} \sqrt{{[ Re ( {ℱ}_{{f}^{sin}} ) ]}^{2} + {[ Re ( {ℱ}_{{f}^{sin}} ) ]}^{2}} \qquad ( 17 )$$

The signal intensity decreases as the harmonic order increases. Components of the applied current frequency and the harmonic order are analyzed. Here, in order to calculate the accurate amplitude of the harmonic order, the fast Fourier transform output may be divided by the signal length to normalize the signal power and be doubled.

Next, a method of obtaining the power spectral density drop (PSDd) ΔP will be described. FIG. 11 shows an example of the power spectral density (PSD) drop calculated by the power spectral drop calculation unit 55. A horizontal axis is frequency (kHz), and a vertical axis is power spectral density (V²/Hz).

In FIG. 11, a difference between the first-order harmonic signal and third-order harmonic signal is denoted by ΔP. ΔP is determined by a magnitude of P of the harmonic order of the fast Fourier transform (FFT), and the magnitude depends on the electrolyte concentration distribution and the blood protein concentration distribution in the specific skin layer. That is, by obtaining the power spectral density drop ΔP, the electrolyte concentration distribution and the blood protein concentration distribution in the specific skin layer can be calculated. The obtained power spectral density drop ΔP is sent to the calibration curve comparison unit 53A.

### (Calibration Curve Comparison Unit 53A)

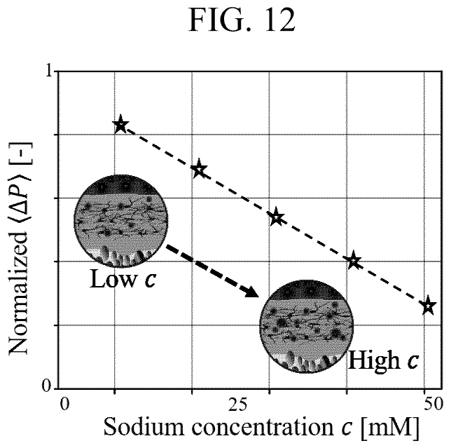

The calibration curve comparison unit 53A calculates at least one of the electrolyte concentration or the blood protein concentration in the specific skin layer based on the power spectral density drop ΔP sent from the power spectral drop calculation unit 55. FIG. 12 shows an example of a relationship between the electrolyte concentration c (a sodium chloride concentration in FIG. 12) and the power spectral density drop ΔP. A horizontal axis of FIG. 12 is an electrolyte concentration (mM), and a vertical axis is a power spectral density drop ΔP[−]. As shown in FIG. 12, since there is a high correlation between the electrolyte concentration c and the power spectral density drop ΔP, the electrolyte concentration in the specific skin layer can be calculated by creating the calibration curve in advance as in the first embodiment.

For example, the conductivity of the dermis layer is analyzed using the power spectral density at different frequencies Pfsin. This is because the stratum corneum layer is such that it can be penetrated by high-frequency injection, and the dermis layer is such that it corresponds to low frequency. As the square-wave response, for example, the low frequency is represented by Pf of the first-order harmonic of f sin=f_{sw}, and the high frequency is represented by Pfsin of the third-order harmonic of f sin=3f_{sw}.

### Third Embodiment

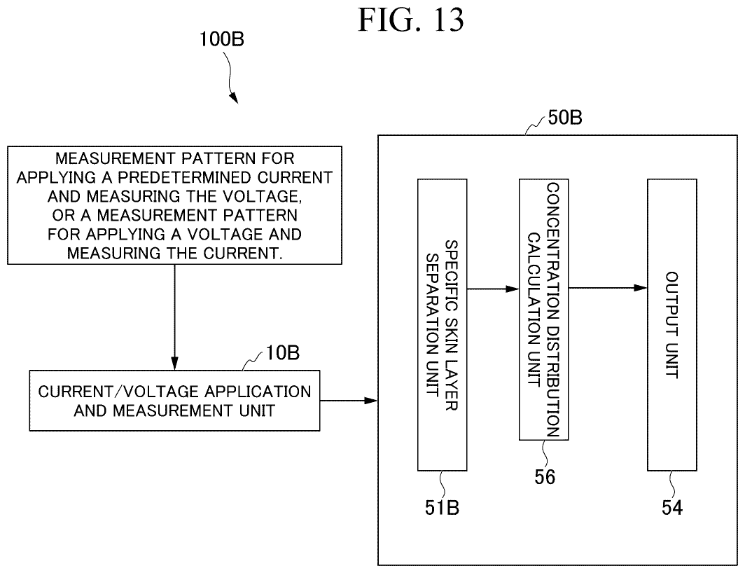

Next, an internal skin measurement apparatus 100B according to a third embodiment of the present invention will be described with reference to FIG. 13.

It is noted that in the second embodiment, the same portions as the constituent elements in the above-described first and second embodiments will be designated by the same reference signs, and a description thereof will be omitted, and only different points will be described.

As shown in FIG. 13, an internal skin measurement apparatus 100B includes a current/voltage injection measurement unit 10B and a concentration distribution measurement unit 50. A concentration distribution measurement unit 50B includes a specific skin layer separation unit 51B, an electrical property distribution calculation unit 56, and the output unit 54.

### (Current/Voltage Injection Measurement Unit 10B)

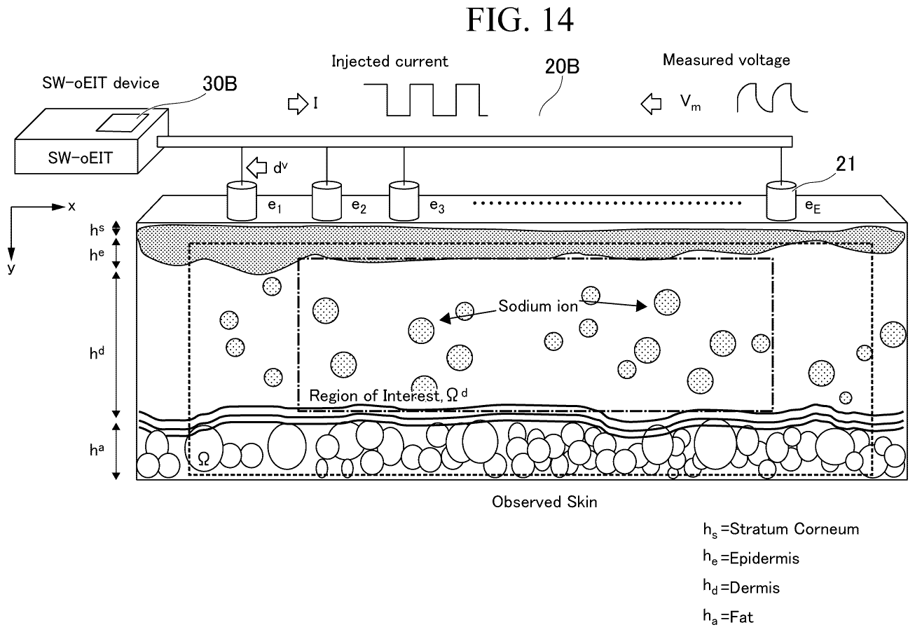

A description will be given of the current/voltage injection measurement unit 10B using FIG. 14. As shown in FIG. 14, the current/voltage injection measurement unit 10B is provided with a sensor 20B and a control unit 30B. The sensor 20B includes a plurality of electrodes 21 (number Q of electrodes) able to be arranged on the skin at intervals from each other. The current/voltage injection measurement unit 10B applies a predetermined current or potential difference between each of the electrodes 21 in a state where the electrodes 21 of the sensor 20B is in contact with the skin of the person to be measured, and measures the potential difference or current. In a case of applying a current, the potential difference is measured based on a predetermined current application/voltage measurement pattern (a pattern in which two electrodes are sequentially selected from a large number of electrodes, a current is applied, and the potential difference is sequentially measured). At this time, it is desirable to also measure the phase (time lag between the applied current and the measured potential difference). In a case of applying a potential difference, the current is measured based on a predetermined voltage application/current measurement pattern (a pattern in which two electrodes are sequentially selected from a large number of electrodes, a potential difference is applied, and the current is sequentially measured). At this time, it is preferable to also measure the phase (time lag between the applied potential difference and the measured current). Below, a description will be given centering on a case in which a current is applied and detailed description of a case in which a potential difference is applied may be omitted. Here, in the third embodiment, the number Q of the electrodes 21 is preferably 8 or more. The number Q of the electrodes 21 is more preferably 16 or more.

FIG. 14 is an example in which the plurality of electrodes 21 are arranged in a single row. The plurality of electrodes 21 may be arranged in a single row on the upper surface of the skin or may be arranged two-dimensionally. In a case where the plurality of electrodes 21 are arranged in a single row, a two-dimensional region of a semicircle in a skin depth direction with a length between a leftmost electrode (e1 in the example of FIG. 14) and a rightmost electrode (eE in the example of FIG. 14) of the electrodes 21 as a diameter can be set as a region of interest, and a two-dimensional image of the specific skin layer can be obtained. In addition, in a case where the plurality of electrodes 21 are arranged two-dimensionally, a three-dimensional region of a hemisphere in the skin depth direction with an outermost electrode of the electrodes 21 in the skin surface as a diameter can be set as a region of Interest, and a three-dimensional image of the specific skin layer can be obtained. The physical quantities of these images are one or more of a conductivity distribution, a permittivity distribution, or a phase distribution in the specific skin layer.

### (Control Unit)

The control unit 30B is provided with, for example, a multiplexer for switching between a current applying electrode that applies a current (or a voltage applying electrode that applies a potential difference) and a voltage measuring electrode that measures a potential difference (or a current measuring electrode that measures a current), an impedance analyzer that performs voltage measurement (or current measurement) and phase measurement, and the like. An impedance analyzer is a component that measures impedance, that is, the ratio of a measured potential difference (applied potential difference) to an applied current (measured current) and the phase thereof by varying the application frequency and amplitude. For example, the control unit 30B executes a predetermined program in the CPU and controls the multiplexer and the impedance analyzer to perform impedance measurement (measurement of the ratio of the potential difference to the current and the phase thereof). The impedance measurement may be performed by controlling the control unit 30B only within the current/voltage injection measurement unit 10B, or the impedance measurement may be performed by controlling the control unit 30B according to a program executed by the concentration distribution measurement unit 50B. The results of the impedance measurement are sent to the concentration distribution measurement unit 50B. The method for transmitting information to the concentration distribution measurement unit 50B is not particularly limited. Information may be sent from the control unit 30B to the concentration distribution measurement unit 50B through a wire, or may be sent to the concentration distribution measurement unit 50B.

The control unit 30B applies a current between the electrodes 21 and measures a potential difference based on a predetermined current application/voltage measurement pattern (a pattern of applying a current between which electrodes and measuring a potential difference between which electrodes). Alternatively, the control unit 30A applies a potential difference between the electrodes 21 and measures a current based on a predetermined voltage application/current measurement pattern. In a case of applying the current and similarly in a case of applying the potential difference, the electrodes 21 between which the current (potential difference) is applied and the electrodes between which the potential difference (current) is measured are not particularly limited. The control unit 30B measures the current and the phase or the potential difference and the phase using two or more electrode pairs in which the inter-electrode distance in the sensor 20 are different. Examples of a measurement method include a two-terminal method, a three-terminal method, a four-terminal method, and a four-terminal quasi-adjacent method.

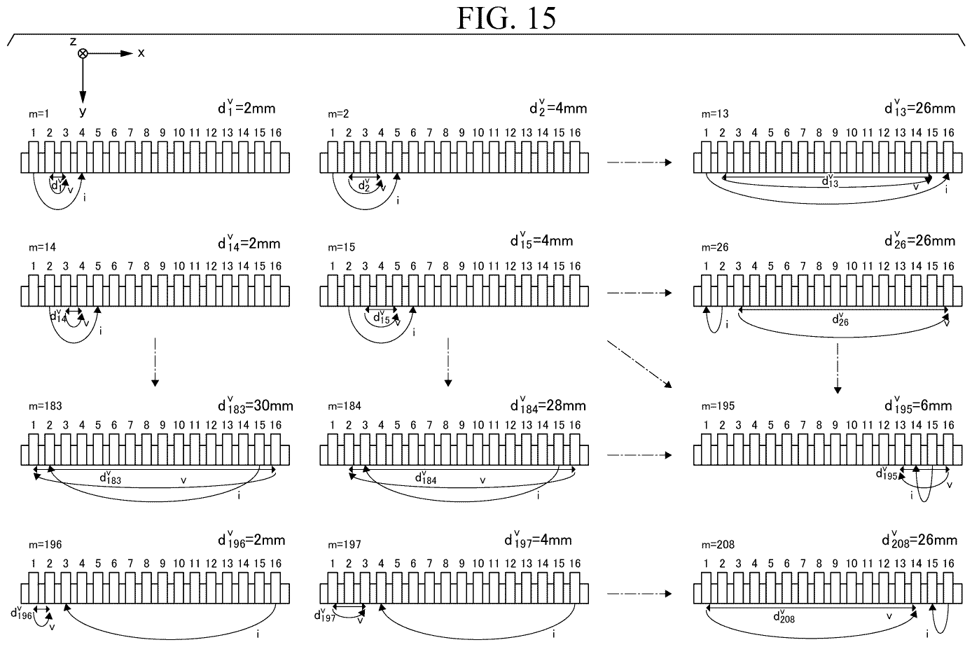

Here, the four-terminal quasi-adjacent method will be described. FIG. 15 is a measurement pattern of the four-terminal quasi-adjacent method in a case where the number of the electrodes 21 is 16. As shown in FIG. 15, in the four-terminal quasi-adjacent method, the measurement is performed such that at least one of the pair of electrodes 21 for current application (current application electrode pair) and the pair of electrodes 21 for potential difference measurement (potential difference measurement electrode pair) are adjacent to each other. In the case of FIG. 15, since the number Q of electrodes is 16, there are a total of 16 types of selection methods for one electrode (current selection electrode) of the current application electrode pair. In addition, one of the potential difference measurement electrode pairs adjacent to the selected current selection electrode is selected as the electrode (potential difference selection electrode) adjacent thereto. The potential difference selection electrode is not changed until the current selection electrode is changed. The potential difference is measured by the potential difference measurement electrode pair (for example, the second electrode and the third electrode) adjacent to the current application electrode pair, excluding the current application electrode pair (for example, the first electrode and the fourth electrode). After the measurement, one electrode of the current application electrode pair is shifted by one (for example, shifted from the fourth electrode toward the sixteenth electrode), and the position of the other electrode of the adjacent potential difference measurement electrode pair is also changed to a position adjacent to the other electrode of the current application electrode pair. Measurement is performed by shifting one electrode of the current application electrode pair to one electrode before the selected electrode. Therefore, there are 13 types of measurement patterns for one current selection electrode. Therefore, in the four-terminal quasi-adjacent method, the total number of measurement types (measurement patterns) M is 208. A square-wave current or the like may be applied as in the second embodiment.

### (Specific Skin Layer Separation Unit)

The specific skin layer separation unit 51B calculates the difference data by performing the difference processing on the potential difference and the phase or the current and the phase measured by the current/voltage injection measurement unit 10B. A penetration depth of the current in the specific skin layer is related to the measurement inter-electrode distance d^{v}, and the penetration depth of the current to the specific skin layer is essential to visualize the concentration distribution of the specific skin layer. In a case where the measurement inter-electrode distance d^{v} is too narrow, the current flows linearly from one electrode to the other electrode, and it is difficult to obtain the impedance information of the specific skin layer at a deep position. On the other hand, in a case where the measurement inter-electrode distance d^{v} is larger than the depth of the specific skin layer, the current flows excessively into the subcutaneous fat layer, and it is difficult to obtain the impedance information of the specific skin layer. In the third embodiment, the specific skin layer separation unit 51B performs predetermined processing based on a spatial voltage threshold d^{Γ} (SVT), amplifies the potential difference (or the current) including the information on the concentration distribution of the target component (electrolyte, blood protein, or the like) in the specific skin layer of interest, and reduces the potential difference (or the current) including the information on the specific skin layer of no interest. Here, the spatial voltage threshold is a threshold value based on the inter-electrode distance, and is a function of the inter-electrode distance. That is, it is possible to focus on the concentration distribution of the target component (electrolyte, blood protein, or the like) in the stratum corneum layer, the epidermis layer, the dermis layer, and/or the fat layer in the specific skin layer according to the inter-electrode distance. The specific skin layer separation unit 51B sends the processed current or potential difference, which is the difference data after performing the predetermined processing on the current or the potential difference using the spatial voltage threshold d^{Γ}, to the electrical property distribution calculation unit 56.

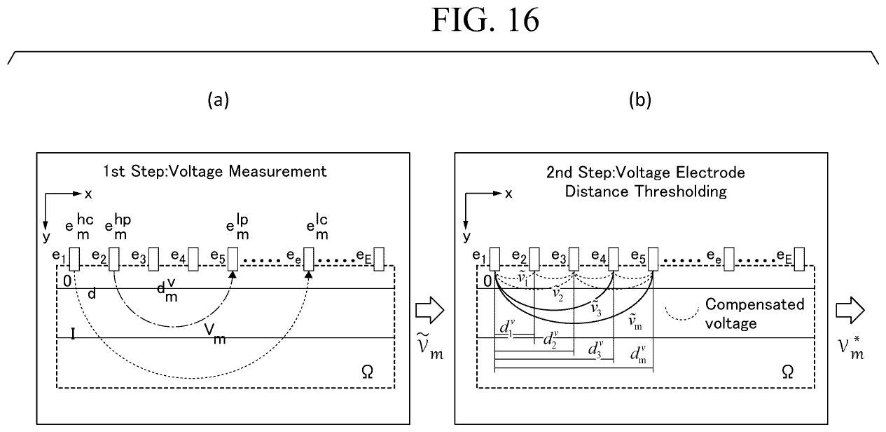

FIG. 16 is a diagram for illustrating processing using a spatial voltage threshold (SVT). FIG. 16A shows a state of the potential difference measurement, and FIG. 16B shows the potential difference after the processing based on the threshold value d^{Γ}. A distance d^{v}ₘ between the potential difference measurement electrodes of FIG. 16 affects the measurement depth. Here, m of d^{v}m means a measurement number. The spatial voltage threshold (SVT) d^{Γ} can be appropriately set according to the target specific skin layer. For example, in a case of acquiring the data of the dermis layer, in d^{v}m≤dΓ, the measurement depth is set to the stratum corneum layer and the epidermis layer. In a case of acquiring the data of the dermis layer, d^{Γ} is, for example, 1 to 6 mm. The spatial voltage threshold (SVT) can be set by examining the influence of the electrolyte concentration in the target specific skin layer in advance. The potential difference v*m after the processing based on the spatial voltage threshold is determined from an average value of v^{\tilde{ }}ₘ, for example, to reduce the influence of the high resistance of the stratum corneum layer in the measurement. v^{\tilde{ }}ₘ is a time average of the potential difference represented by Expression (18). On the other hand, in a case of d>d^{Γ}, since the measurement depth is in the deep portion (dermis layer or subcutaneous fat tissue), v*m after the processing with the spatial voltage threshold is equal to v^{\tilde{ }}ₘ. In summary, the potential difference (compensation potential difference) v*ₘ after the processing with the spatial voltage threshold is represented by Expression (19).

Next, an example will be described in which the distance between adjacent electrodes is defined as d, 16 electrodes 21 are arranged, and the measurement is performed by the four-terminal method using the application electrode pair and the measurement electrode pair, with the four-terminal quasi-adjacent method as the measurement method between the electrodes. More specifically, in each measurement number m, a constant current of the square-wave is applied to the application electrode pair (high current application electrode e^{hc}ₘ and low current application electrode e^{lc}ₘ), and the measurement potential difference is measured from the measurement electrode pair (high potential electrode e^{hp}ₘ and low potential electrode e^{lp}ₘ). For example, the high potential electrode e^{hp}ₘ is expressed by integer division as represented in Expression (20) below. m∈{1, 2, . . . , 208} in Expression (20 is a measurement number. The low potential electrode e^{lp}ₘ is expressed by integer division and modulus division as represented by Expression (21). The distance d^{v}ₘ (mm) of the electrode voltage is determined as represented by Expression (22) from the distance between e^{hp}ₘ and e^{lp}ₘ.

$${\tilde{v}}_{m} = \sqrt{\frac{1}{T} {\int }_{0}^{T} {{v}_{m} ( t )}^{2} dt} \qquad ( 18 )$$

$${v}_{m}^{*} = \{ \begin{matrix}\frac{1}{M} {\sum }_{m}^{M} {\tilde{v}}_{m} , & if {d}_{m}^{v} \leq {d}^{Γ} \\ {\tilde{v}}_{m} , & if {d}_{m}^{v} > {d}^{Γ}\end{matrix} \qquad ( 19 )$$

$${e}_{m}^{hp} = ⌊ m / 13 ⌋ + 1 \qquad ( 20 )$$

$${e}_{m}^{lp} = ⌊ m / 13 ⌋ + ⌊ m mod 13 ⌋ \qquad ( 21 )$$

$${d}_{m}^{v} = \vert {e}_{m}^{hp} - {e}_{m}^{lp} \vert d \qquad ( 22 )$$

### (Electrical Property Distribution Calculation Unit)

The electrical property distribution calculation unit 56 calculates at least one of a conductivity distribution, a permittivity distribution, or a phase distribution based on the processed current or potential difference sent from the specific skin layer separation unit 51B. The calculated electrical property distribution is sent to the output unit 54.

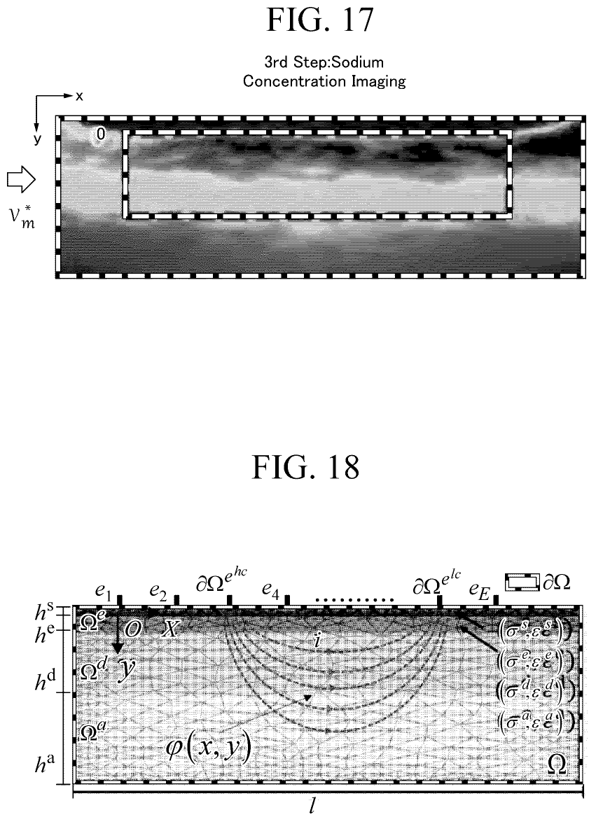

FIG. 17 shows an example of the image reconstruction. The sodium ion concentration distribution image of the skin region Ω is reconstructed based on the conductivity distribution σ represented by Expression (23), and in this example, the sodium ion concentration distribution of the dermis layer within the dotted line is emphasized by the predetermined processing based on the spatial voltage threshold d^{Γ} (SVT). Here, rₙ in Expression (23) is represented by Expression (24) and is a row vector representing a spatial position of an n-th (1≤n≤N) mesh element in a case where the skin region Ω is divided into N pieces of mesh elements.

By obtaining a Jacobian matrix (sensitivity matrix) J consisting of all combinations of the current injection I and the measurement voltage v on each electrode in advance, the conductivity distribution σ, the permittivity distribution, and the phase distribution of the skin region Ω can be obtained. For example, the Jacobian matrix J of the skin region Ω is represented by Expression (25). Here, m is defined as a measurement pattern (1≤m≤M), M is defined as the total number of measurement patterns, n is defined as a spatial position (mesh element) (1≤n≤N), N is defined as the total number of meshes of the spatial resolution, and T is defined as a transpose matrix. In a case where the imaging of the conductivity distribution by the potential difference measurement is taken as an example, the Jacobian matrix Jₘₙ of the n-th mesh element in the m-th measurement pattern can be defined by the second term of Expression (26), and can be defined by a change rate of the measurement potential difference vm in the m-th (1≤m≤M) measurement pattern in a case where the conductivity σₙ of the n-th (1≤n≤N) mesh element is changed (expressed by a partial differential symbol), and J is a coefficient of the conductivity distribution σ and the measurement potential difference v. Specifically, J can be calculated by, for example, the third term of Expression (26). Here, in a case where a combination of electrodes for current application is denoted by k and a combination of electrodes for potential difference measurement is denoted by l in the measurement pattern m, φ(I^{k}) in Expression (26) is a potential of the n-th mesh element affected by the current I injected between the electrodes of k, φ(I^{l}) is a potential of the n-th mesh element in a case where the current I is temporarily injected between the electrodes of l, ∇ is a nabla partial differential operator, and the integral symbol indicates an integral over the entire skin region Ω. The Jacobian matrix (sensitivity matrix) J corresponds to the electrolyte and the blood protein as the target component, and further corresponds to the application frequency, and by preparing different J in advance for each, a more accurate image can be obtained.

$$σ = {[ {σ}_{1} ( {r}_{1} ) , … , {σ}_{n} ( {r}_{n} ) , … , {σ}_{N} ( {r}_{N} ) ]}^{T} ∈ {ℝ}^{N} \qquad ( 23 )$$

$${r}_{n} = ( {x}_{n} , {y}_{n} ) ∈ {ℝ}_{2} \qquad ( 24 )$$

$$\begin{matrix}J = [ {J}_{1} , {J}_{2} , … , {J}_{n} , … {J}_{N} ] ∈ {ℝ}^{M \times N} \\ {J}_{n} = [ {J}_{1} , {J}_{2} , … , {J}_{m} , … {J}_{M} ] ∈ {ℝ}^{M}\end{matrix} \} \qquad ( 25 )$$

$${J}_{mn} = \frac{\partial {v}_{m}}{\partial {σ}_{n}} {\int }_{Ω} ∇ φ ( {I}^{k} ) \cdot ∇ φ ( {I}^{l} ) d Ω \qquad ( 26 )$$

The conductivity distribution of the skin layer is calculated using a Gauss-Newton method (Expression (27)). Here, R in Expression (27) is a regularization matrix, and λ is a relaxation coefficient scalar automatically determined by an L curve method (Hansen and O'Leary 1993). Δv in Expression (27) is represented by Expression (28). Δvm in Expression (28) is a normalized measurement voltage under a skin boundary shape ∂Ω and is represented by Expression (29). Here, vm(0) is an initial measurement voltage, and vm(c) is an intermediate voltage based on the target component concentration c. The electrical property distribution calculation unit 56 sends the electrical property distribution of the target component obtained above to the output unit 54.

### (Boundary Processing)

The electrical property distribution calculation unit 56 may further perform the boundary processing on the calculated electrical property distribution. Hereinafter, the boundary processing will be described.

FIG. 18 is a schematic diagram of each specific skin layer to be used in the electromagnetic simulation. h^{s} in FIG. 18 is a thickness of the stratum corneum layer, h^{e} is a thickness of the epidermis layer (excluding the stratum corneum layer), h^{d} is a thickness of the dermis layer, h^{a} is a thickness of the fat tissue layer, Ω^{e} indicates a target region of the epidermis layer, Ω^{d} indicates a target region of the dermis layer, Ω^{a} indicates a target region of the fat tissue layer, σ^{s} indicates a conductivity of the stratum corneum layer, and ε^{s} indicates a relative permittivity of the stratum corneum layer. σ^{e} indicates a conductivity of the epidermis layer, and ε^{e} indicates a relative permittivity of the epidermis layer. σ^{d} indicates a conductivity of the dermis layer, and ε^{e} indicates a relative permittivity of the dermis layer. σ^{a} indicates a conductivity of the fat tissue layer, and ε^{a} indicates a relative permittivity of the fat tissue layer. φ (x,y) is a potential at coordinates (x,y).

The current applied from the electrode e^{k} (k=1, . . . , E) of FIG. 18 generates an electric field E in the target region Ω, and the current density i of the target region Ω is represented by i=σE. A physical relationship between the conductivity distribution in the target region Ω and the boundary voltage is governed by a partial differential equation derived from the Maxwell equation of Expression (30). In a case where the current I is applied through the electrode e^{k} (e₁, e₂, . . . , e_{E} in FIG. 18) on the skin surface ∂Ω, the potential φ of the target region Ω is solved from Expression (31). For example, the electrical property distribution can be more accurately calculated by performing the calculation based on the Neumann boundary conditions of Expressions (32) and (33). Here, n represents an outer unit normal vector, ∂Ω^{ek} represents an electrode boundary, and dS represents a minute area element of the corresponding electrode boundary.

$${σ}^{k + 1} = {σ}^{k} + {( {J}^{T} J + λ R )}^{- 1} {J}^{T} Δ v \qquad ( 27 )$$

$$Δ v = {[ Δ {v}_{1} , … , Δ {v}_{m} , … Δ {v}_{M} ]}^{T} ∈ {ℝ}^{M} \qquad ( 28 )$$

$$Δ {v}_{m} = {v}_{m} ( c ) - {v}_{m} ( 0 ) \qquad ( 29 )$$

$$∇ \cdot ( σ ∇ φ ) = 0 \qquad ( 30 )$$

$$φ ( x , y ) + σ ( x , y ) \frac{\partial φ ( x , y )}{\partial n} = {v}_{k} , ( x , y ) ∈ {e}_{k} \qquad ( 31 )$$

$${\int }_{\partial {Ω}^{{e}_{k}}} σ ( x , y ) \frac{\partial φ ( x , y )}{\partial n} dS = I , ( x , y ) ∈ \partial {Ω}^{{e}_{k}} \qquad ( 32 )$$

$$σ ( x , y ) \frac{\partial φ ( x , y )}{\partial n} = 0 on \partial Ω excluding \partial {Ω}^{{e}_{k}} \qquad ( 33 )$$

The internal skin measurement apparatuses 100, 100A, and 100B according to the present embodiment have been described above.

Note that, the technical scope of the present invention is not limited to the above embodiment and it is possible to make various changes thereto in a range not departing from the spirit of the present invention.

In addition, it is possible to appropriately replace the constituent elements in the embodiments with well-known constituent elements in a range not departing from the spirit of the present invention, and such modifications may be combined as appropriate.

### EXAMPLE

Hereinafter, the present invention will be described with reference to Examples, but the present invention is not limited to Examples.

### Example 1

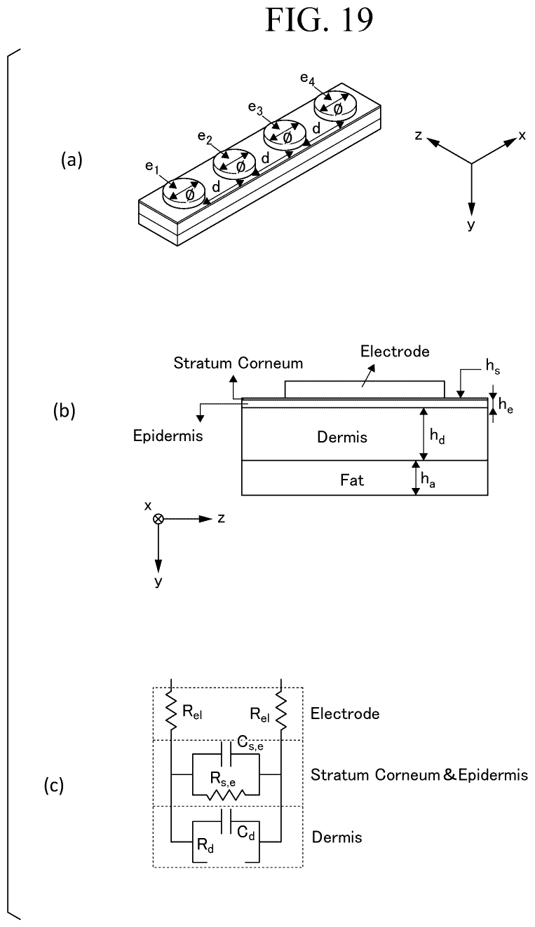

To optimize the measurement parameters (the ratio r(r=φ/h_{d}) of the electrode diameter φ to the dermis layer thickness h_{d}, and the inter-electrode distance d) of the first embodiment, two electromagnetic simulations were performed assuming changes in the concentration of ions and proteins in each actual layer. FIG. 19A shows the sensor used in the two electromagnetic simulations, and four non-invasive electrodes were arranged on the skin surface. FIG. 19B shows each configuration of the specific skin layer, and the specific skin layer is composed of a stratum corneum layer, an epidermis layer, a dermis layer, and a fat layer. The thickness of each layer is represented by hs for a stratum corneum layer, he for an epidermis layer, hd for a dermis layer, and ha for a fat layer. In the condition of the electromagnetic simulation, the thickness of the dermis layer was varied, and the thickness of each skin layer excluding the dermis layer was fixed, specifically, hs for the stratum corneum layer was 0.05 mm, he for the epidermis layer was 0.45 mm, and ha for the fat layer was 5 mm. The application constant current to the non-invasive electrodes 1 to 3 (e₁, e₂, and e₃) was I_{hc}=1 mA, and the frequency thereof was f=100 Hz to 1 MHz. On the other hand, the el4 of the electrode 4 is always set as the earth, and for example, in a case where a current is applied between the non-invasive electrode 1 and the non-invasive electrode 4, the other non-invasive electrode 2 and the non-invasive electrode 3 function as floating electrodes, and the current does not flow to the non-invasive electrode 2 and the non-invasive electrode 3. FIG. 19C shows an equivalent circuit of the specific skin layer simplified by the two-terminal method. Here, the current flows in the specific skin layer present between the non-invasive electrodes under the influence of the size of the electric equivalent circuit of the combination of the capacity component C_{s,e} of the stratum corneum layer and the epidermis layer, the resistance component R_{s,e} of the stratum corneum layer and the epidermis layer, the capacity component C_{d} of the dermis layer, and the resistance component R_{d} of the dermis layer.

Electromagnetic simulation (estimated distribution function γ\hat{ }(ln τ) when variation of ratio r and inter-electrode distance d)

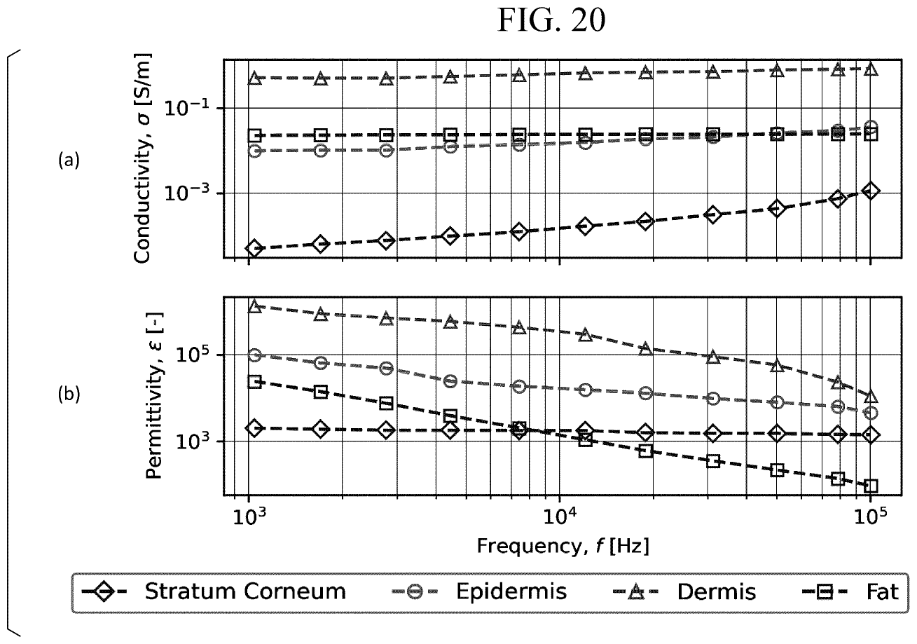

The penetration depth of EFCL into the specific skin layer depends on the ratio r (r=φ/h_{d}) of the electrode diameter φ to the dermis layer thickness h_{d} and the distance between adjacent electrodes d. The variation of r was set to 0.5 (φ/h_{d}=0.5/1), 0.7 (φ/h_{d}=1/1.5), 1 (φ/h_{d}=2/2), and 1.2 (φ/h_{d}=3/2.5), and the inter-electrode distance d was set to 2 mm to 8 mm. The electromagnetic simulation was executed using the non-invasive electrode (e₁, e₂, or e₃) for current application, the ratio r, and the inter-electrode distance d as parameters to examine the optimal electrode diameter and the optimal inter-electrode distance. FIG. 20 shows the frequency dependence of the known conductivity σ and the known relative permittivity ε in each specific skin layer used under the electromagnetic simulation condition. A vertical axis of FIG. 20A shows a conductivity (S/cm), and a horizontal axis of FIG. 20A shows an applied current frequency (Hz). A vertical axis of FIG. 20B shows a relative permittivity (−), and a horizontal axis of FIG. 20B shows an applied current frequency (Hz).

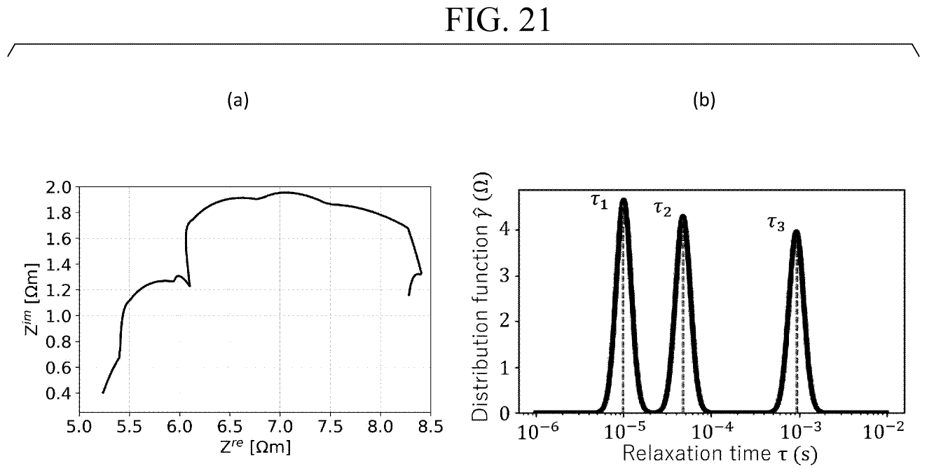

FIG. 21 is an electromagnetic simulation result in the specific skin layer separation unit in a case of r=1 (φ/h_{d}=2/2) and an inter-electrode distance d=1 mm under the electromagnetic simulation condition described above, FIG. 21A shows a Nyquist plot obtained from the electromagnetic simulation result of the impedance, and FIG. 21B shows the estimated distribution function γ\hat{ }(ln τ) at a specific relaxation time τ, which is an output result of the distribution function estimation unit 52 according to the embodiment of the present invention. These three peaks occurred in a relaxation time τ of 10₋₅ to 10₋₃ seconds. The contribution of each layer of the skin to the relaxation time was examined below.

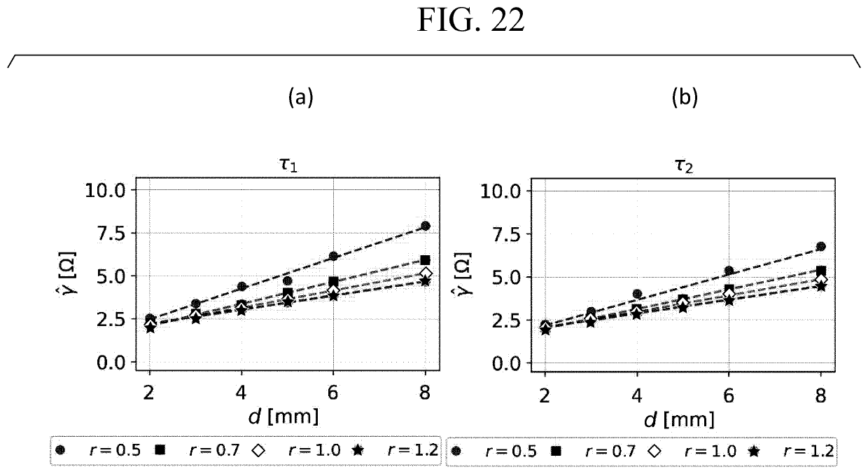

FIG. 22 shows a change in the estimated distribution function γ\hat{ }(ln τ) when the inter-electrode distance d is varied with the ratio r(=φ/h_{d}) as a parameter, and as an example, FIG. 22A shows the estimated distribution function γ\hat{ }(ln τ) at τ1, and a horizontal axis is the inter-electrode distance d. A vertical axis of FIG. 22B is the estimated distribution function γ\hat{ }(ln τ) at τ2, and a horizontal axis is the inter-electrode distance d. From these diagrams, it was found that, in a case of r=0.5, the estimated distribution function γ\hat{ }(ln τ) was larger than in other ratios. This means that the smaller the electrode diameter φ than the dermis layer thickness hd, the larger the γ\hat{ }(ln τ) was. Furthermore, as the inter-electrode distance d increased, the magnitude of γ\hat{ }(ln τ) also increased. It is considered that this was because the electrode diameter φ was comparable to the dermis layer thickness hd.

### Example 2

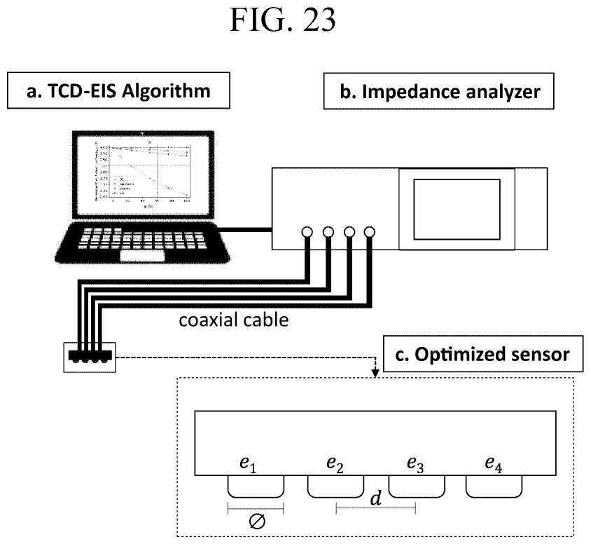

Next, to experimentally demonstrate the first embodiment using the measurement parameters (the ratio r and the inter-electrode distance d) of the first embodiment optimized in Example 1, an experiment for the specific skin layer separation unit was performed using pig skin tissue (fat layer and muscle were also attached) having substantially the same mechanical and electrical characteristics as human skin. FIG. 23 shows an outline of the experimental apparatus. FIG. 23 is composed of a sensor, a four-channel multiplexer, an impedance analyzer (IM 3570, HIOKI E.E. CORPORATION, Tokyo, Japan) as a data acquisition system, and a personal computer for instruction and post-processing. As the sensor, a printed circuit board (PCB) having dimensions of 25 mm×35 mm×1.6 mm with four non-invasive electrodes arranged in a 1×4 arrangement was used.

The non-invasive electrode was made of stainless steel, had a diameter of 1 mm, and had a center-to-center distance of 1 mm between adjacent electrodes and a thickness of 1 mm from the printed circuit board.

The current applied to the measurement was I=1 mA, and the swept applied current frequency was f=100 Hz to 1 MHz. In the present example, the two-terminal method was used as a method of measuring the impedance, and the relationship between the measurement number and each non-invasive electrode is shown in Table 1. The column of Hc in Table 1 indicates the electrode number for high current application, the column of Hp indicates the electrode number for high potential measurement, the column of Lc indicates the electrode number for low current application, and the column of Lp indicates the electrode number for low potential measurement. The current flowed from the high current application electrode (Hc) through the pig skin tissue and reached the low current application electrode (Lc) including the ground. The electrode numbers e₁ (abbreviated as 1 in the table), e₂ (abbreviated as 2 in the table), and e₃ (abbreviated as 3 in the table) functioned as the Hc and Hp non-invasive electrodes, and the electrode number e₄ (abbreviated as 4 in the table) always functioned as the Lp and Lc (grounded) electrodes. By the measurement of Table 1, a total of three measurement values were obtained.

The pig skin tissue sample was composed of a stratum corneum layer, an epidermis layer, a dermis layer, a fat layer, and a muscle layer, and the temperature was adjusted to reach an appropriate temperature (±35° C.). To simulate the change in the sodium ion concentration of the skin, four aqueous sodium chloride (NaCl) solutions having different concentrations (20 mM, 30 mM, 35 mM, and 40 mM) were prepared and injected into the dermis layer of the pig. As a hyperparameter, λ=10⁻⁶ was set, and the potential difference was measured based on the measurement sequence of Table 1.

TABLE 1

| | |
|---|---|
| Measurement | Electrode number |

| number | H_{c} | Hₚ | Lₚ | L_{c} |
|---|---|---|---|---|
| 1 | 1 | 1 | 4 | 4 |
| 2 | 2 | 2 | 4 | 4 |
| 3 | 3 | 3 | 4 | 4 |

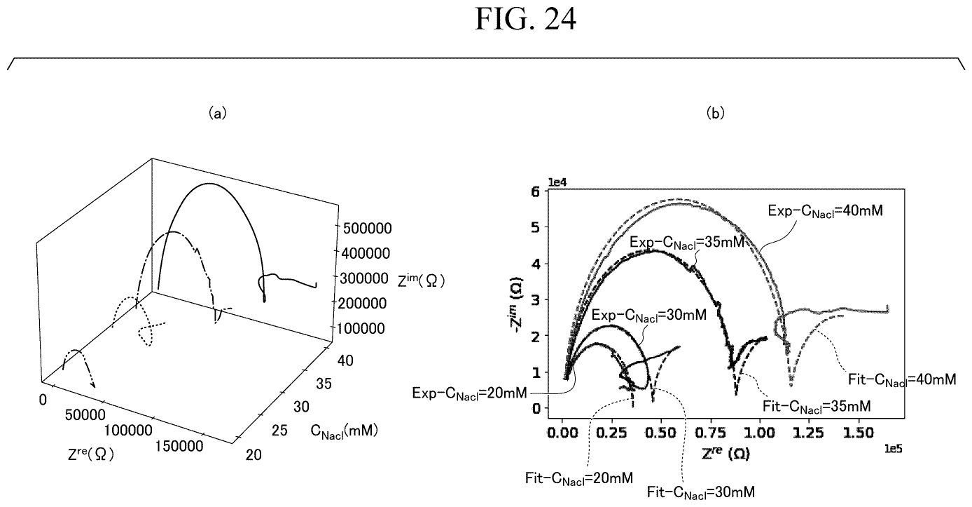

FIG. 24A shows a result of a Nyquist plot in which the sodium chloride aqueous solution concentration was used as a parameter (y-axis), the applied current frequency was swept, and the real part (x-axis) and the imaginary part (z-axis) of the impedance were used. FIG. 24B shows a comparison between the experimental result (solid line) and the equivalent circuit fitting (dotted line) based on the electrical equivalent circuit (EEC). As shown in FIG. 24B, the fitting between the experimental result and the equivalent circuit was well matched. In addition, it was confirmed that the resistance value Z^{re} obtained from the value of the x-axis indicating the minimum value of the y-axis of the Nyquist plot increased with an increase in the sodium chloride aqueous solution concentration c.

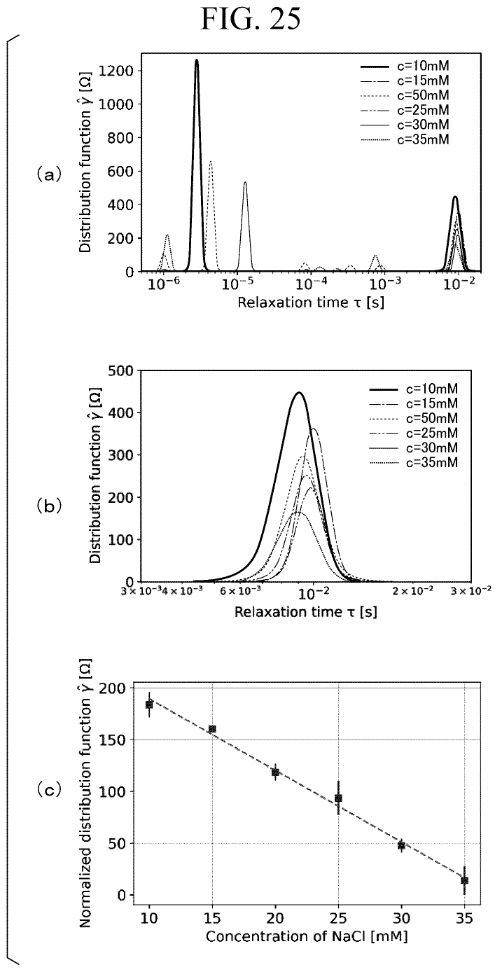

FIG. 25A shows a calculation result of the specific skin layer separation unit of the first embodiment of the experiment using the pig skin tissue, and shows a relationship between the relaxation time τ and the estimated distribution function γ\hat{ }(ln τ) in a case where the sodium chloride aqueous solution concentration was used as a parameter. From this figure, several peak values were confirmed. These peak values correspond to the distribution function for each specific skin layer. The estimated distribution function γ\hat{ }(ln τ) for the dermis layer is a peak value of the relaxation time τ=10⁻² seconds. FIG. 25B shows a normal distribution function in which the relaxation time τ is 10⁻² seconds and the estimated distribution function γ\hat{ }(ln τ) based on the experiment of the specific skin layer separation unit of the first embodiment, with respect to the change in the sodium chloride aqueous solution concentration. A vertical axis of FIG. 25B is the estimated distribution function γ\hat{ }(ln τ) (Ω), and a horizontal axis is the relaxation time τ (s).

A local maximal value of the estimated distribution function γ\hat{ }(ln τ) calculated using the specific skin layer separation unit 51 of the first embodiment indicates the characteristics of each layer of the specific skin layer. To separate the electrical characteristics of the dermis layer from the other epidermis layer, the difference between the adjacent electrode pair and a distant electrode pair was obtained. In a region of a long relaxation time (τ=10⁻² [s]), the peaks were well distinguished, but in other relaxation time regions, the peaks were irregular. From FIG. 25A, it was found that in the high relaxation time region, the distribution function provided information related to the concentration of the sodium ions injected into the dermis layer. Therefore, the analysis was performed focusing on the high relaxation time region in which useful information of the dermis layer was obtained.

FIG. 25B shows an enlarged view of the distribution function in a time constant range near τ=10⁻² [s]. A horizontal axis of FIG. 25B is the sodium chloride aqueous solution concentration, and a vertical axis is the estimated distribution function γ\hat{ }(ln τ). From this figure, it was found that the amplitude decreased as the concentration increased in this region. FIG. 25C shows the estimated distribution function γ\hat{ }(ln τ) calculated by the specific skin layer separation unit of the first embodiment with respect to the change in the sodium chloride aqueous solution concentration near τ=10⁻² s, and linearity was observed. From the above results, it was found that, for example, the sodium ion concentration of the dermis layer could be measured by the internal skin measurement apparatus according to the present embodiment, and the state of edema, the state of necrosis of cells or blood vessels, and the state of chronic kidney disease could be detected.

### Example 3

To experimentally demonstrate the second embodiment, the measurement was performed under the condition (simulating a patient with chronic kidney disease (CKD)) in which the dermis layer electrolyte concentration of the pig skin was changed from 5 mmol/L to 50 mmol/L.

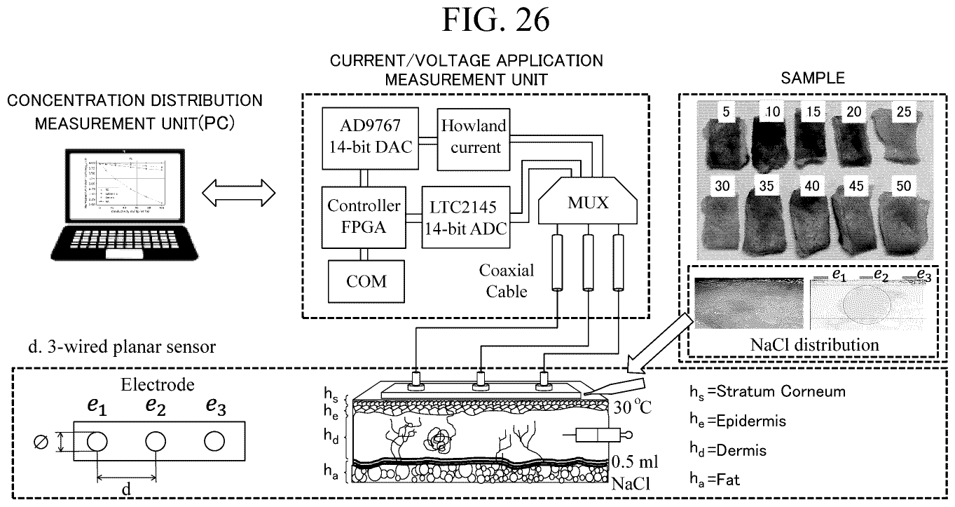

FIG. 26 shows a configuration of the experimental apparatus used in Example 3. As the square-wave applied current device, an FPGA (Xilinx Zynq-SOC) and a three-channel analog multiplexer (ADG1404) were used. The surface of the pig skin tissue was connected to three electrodes e1, e2, and e3 with an inter-electrode distance (non-invasive inter-electrode distance) d=2 mm and an electrode diameter φ=0.8 mm. The electrode e1 was set as a low current application electrode and a low voltage measurement electrode, and the measurement was performed by switching the electrode e2 to a high voltage measurement electrode and the electrode e3 to a high current application electrode. An analog-digital converter LTC2145 was used to acquire a high-speed data signal having a data resolution of 14 bits and a sampling rate of 125 MS/s. The measured potential difference v was transmitted to the power spectral drop calculation unit.

In Example 3, the conductivity of the dermis layer was analyzed in the time domain and the frequency domain based on the potential difference v measured by the two-pair two-electrode method (potential difference measurement between e2 and e1 and current application between e3 and e1). The time-domain analysis includes a maximum measurement potential difference vmax and a charging time, resulting from the resistive and capacitive characteristics of the dermis layer. On the other hand, the frequency-domain analysis includes the power spectral density in the harmonic component. To ensure the reproducibility of the measurement, the measurement was performed 10 times, and the average value was obtained.

The magnitude i of the current was 1.0 mA, the duty cycle δ was 50%, and the applied current frequency f was 1 kHz to 200 kHz. The pig skin tissue was cut into 2×4 cm, and 0.5 mL of an aqueous sodium chloride solution (NaCl+H₂O) was directly injected into the dermis layer by injection. The aqueous sodium chloride solution was prepared by dissolving NaCl powder in 50 mL of distilled water using Expression (34).

$$c = \frac{{m}_{NaCl}}{V \cdot {M}_{r}} \qquad ( 34 )$$

The dermis layer was set to a total of 10 types of sodium chloride aqueous solution concentrations of 5, 10, 15, 20, 25, 30, 35, 40, 45, and 50 [mmol/L]. Here, m^{NaCl} in Expression (34) is a mass of the NaCl powder, V is an amount of the distilled water, and Mr is a relative molecular weight of NaCl and is 58.44 [g/mol]. It was confirmed that the injection of NaCl was uniformly distributed in the dermis layer and did not diffuse into the stratum corneum layer, the epidermis layer, and the fat layer.

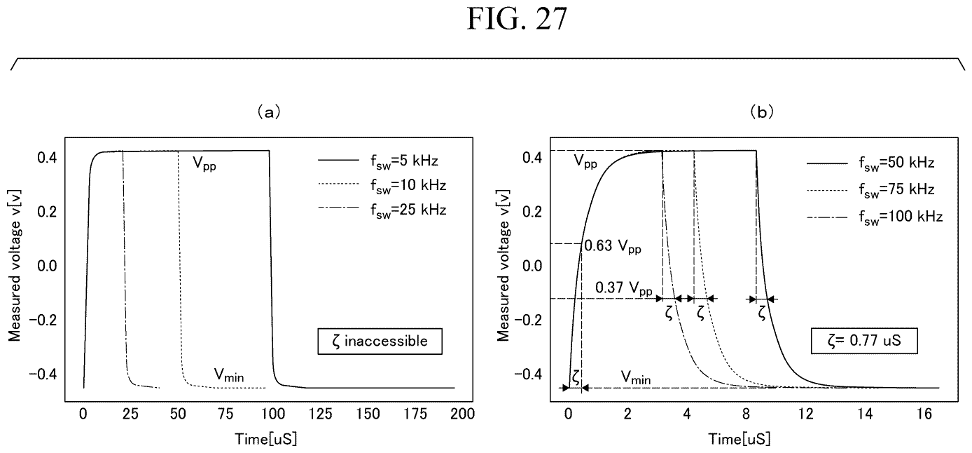

In the square-wave measurement, the time constant τ in the temporal change of the potential to be measured greatly affects the result. Therefore, it is important to determine the appropriate applied current frequency of the square-wave by using the time constant analysis. FIG. 27 shows a measurement result of the measurement potential difference v depending on difference in the applied current frequency f_{sw} in a case where the dermis layer was injected with the sodium chloride aqueous solution concentration c of 10 mmol/L. FIG. 27A shows the results of f_{sw}=5 kHz to 25 kHz. A horizontal axis of FIG. 27A shows an elapsed time (μs), and a vertical axis shows the measured potential difference v(V). FIG. 27B shows the results of f_{sw}=50 kHz to 100 kHz. A horizontal axis of FIG. 27B shows an elapsed time (μs), and a vertical axis shows the measured potential difference v(V). In a case of the applied current frequency of 50 kHz to 100 kHz, it was found that all the applied current frequency ranges were the same time constant value of ζ=0.77 μs in a case where the time constant τ was calculated using Expression (16).

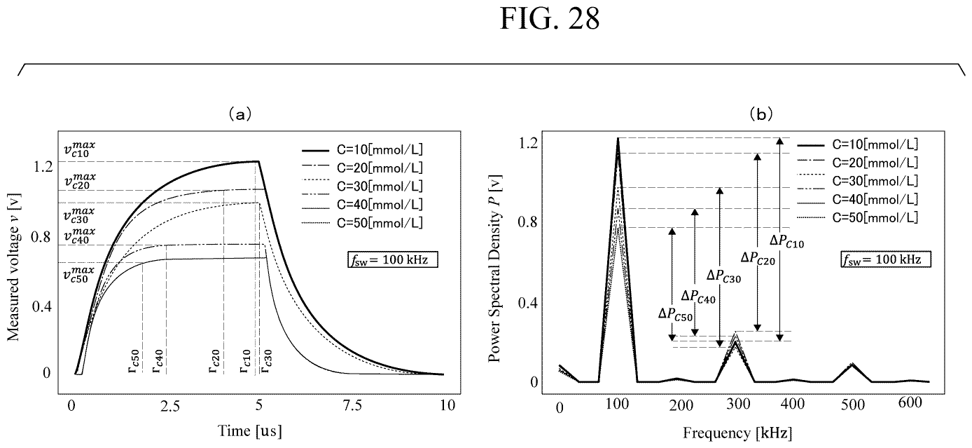

FIG. 28 shows a measurement result of the measurement voltage v depending on difference in the sodium chloride aqueous solution concentration c at the applied current frequency f_{sw}=100 kHz. FIG. 28A shows the result in the time domain, and a horizontal axis is time (μs), and a vertical axis is the measured potential difference v. In the result in the time domain, information related to the maximum measurement voltage vmax and the time constant τ can be acquired. The time constant is an important parameter for calculating the cell content volume, the extracellular resistance, and the intracellular resistance of the dermis layer. From FIG. 28A, it was found that the maximum measurement voltage vmax decreased as c increased, and thus c and the maximum measurement voltage vmax had a linear correlation.

FIG. 28B shows the frequency domain, and shows information related to the power spectral density of the harmonic order.

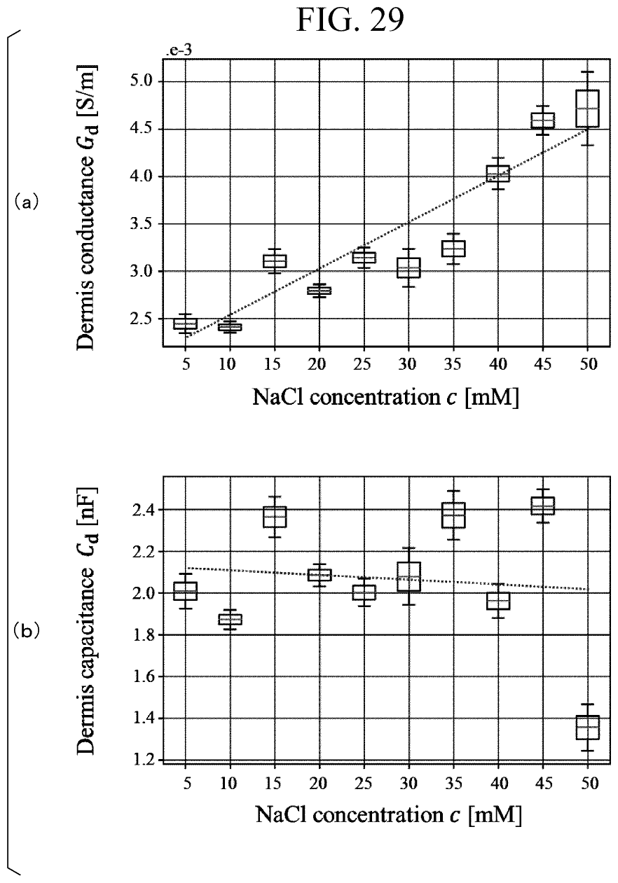

The characteristics of the dermis layer were calculated based on the potential difference v measured by the time-domain analysis. FIG. 29 shows the conductance and the capacitance of the dermis layer based on the time constant analysis of the sodium chloride aqueous solution concentration. A vertical axis of FIG. 29A shows the conductance (S/m) of the dermis layer, and a horizontal axis shows the sodium chloride aqueous solution concentration (mM). A vertical axis of FIG. 29B shows the capacitance (nF) of the dermis layer, and a horizontal axis shows the sodium chloride aqueous solution concentration. From these results, it was confirmed that the conductance in the dermis layer increased with an increase in the sodium chloride aqueous solution concentration in the dermis layer.

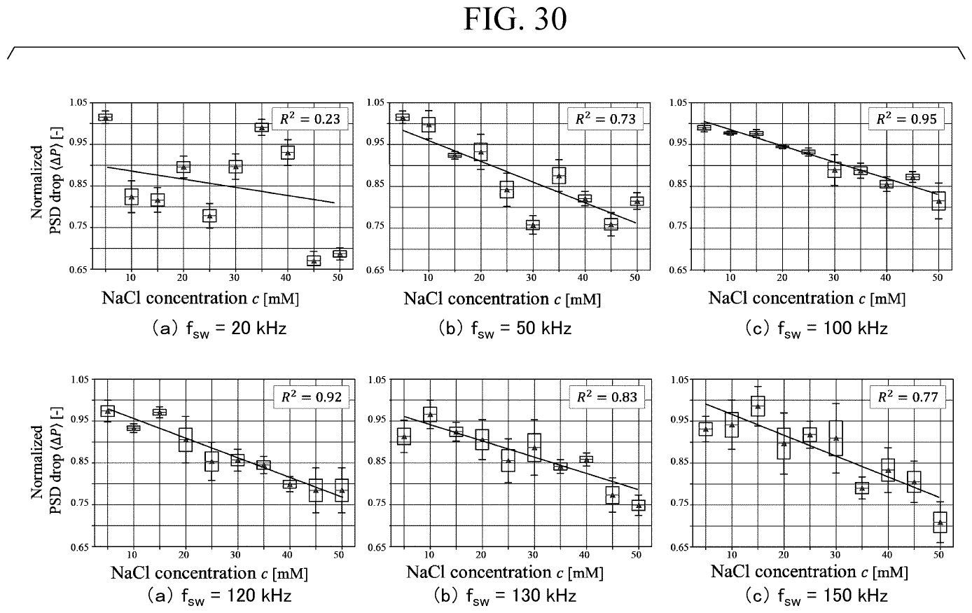

FIG. 30 shows a relationship between c and ΔP in the measurement of the dermis layer of the pig skin using the square-wave applied current f_{sw}=25 kHz to 150 kHz. A vertical axis of FIG. 30 shows the normalized power spectral density drop, and a horizontal axis shows the concentration of the aqueous NaCl solution injected into the dermis layer. In the applied current frequency of 100 kHz and 120 kHz, the coefficient of determination R² was more than 0.90. The coefficient of determination decreased at the applied current frequency f exceeding 100 kHz. Based on the linear regression of the experimental results, the power spectral density drop ΔP was calculated based on Expression (35). On the other hand, ΔP was calculated based on Expression (36) from the calculation. Here, k is a calibration coefficient.

$$Δ P = k ( - 0.0038 c + 1.028 ) \qquad ( 35 )$$

$$Δ P = k ( - 0.0055 c + 1.023 ) \qquad ( 36 )$$

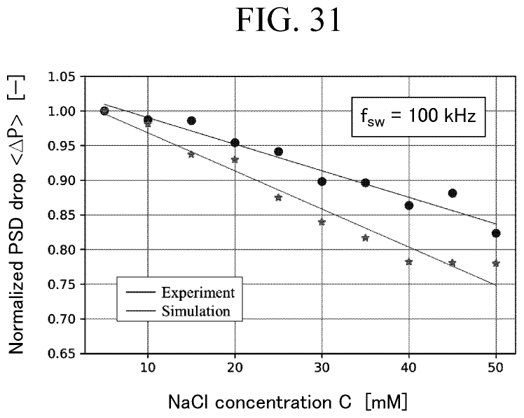

FIG. 31 shows a relationship between the sodium chloride aqueous solution concentration and the normalized PSD drop ΔP at f_{sw}=100 kHz. The harmonic order from the square-wave applied current includes valuable information extracted to represent the conductivity, the permittivity, and the phase of the dermis layer. It was confirmed that the power spectral density drop calculated by subtracting the basic applied current frequency and the voltage amplitude of the third harmonic was in a linear relationship with the conductivity, the permittivity, and the phase that changed in the dermis layer. Table 2 is a result of the time constant analysis for determining the electrical characteristics of the dermis layer. The result of the time constant analysis indicated that the sodium ion concentration c was directly related to the dermis conductance Gd, and the Gd increased as c increased.

TABLE 2

| c | ζ | ν^{max} | G_{d·10}_{^{−3}} | C_{d} |
|---|---|---|---|---|
| [mM] | [μs] | [volt] | [S/m] | [pF] |
| 5 | 0.81 | 0.244 | 2.46 | 1.99 |
| 10 | 0.77 | 0.248 | 2.42 | 1.87 |
| 15 | 0.75 | 0.192 | 3.13 | 2.35 |
| 20 | 0.74 | 0.214 | 2.81 | 2.08 |
| 25 | 0.63 | 0.190 | 3.16 | 1.99 |
| 30 | 0.67 | 0.196 | 3.07 | 2.06 |
| 35 | 0.72 | 0.184 | 3.27 | 2.35 |
| 40 | 0.48 | 0.148 | 4.06 | 1.95 |
| 45 | 0.52 | 0.130 | 4.62 | 2.40 |
| 50 | 0.28 | 0.126 | 4.77 | 1.34 |

### Example 4

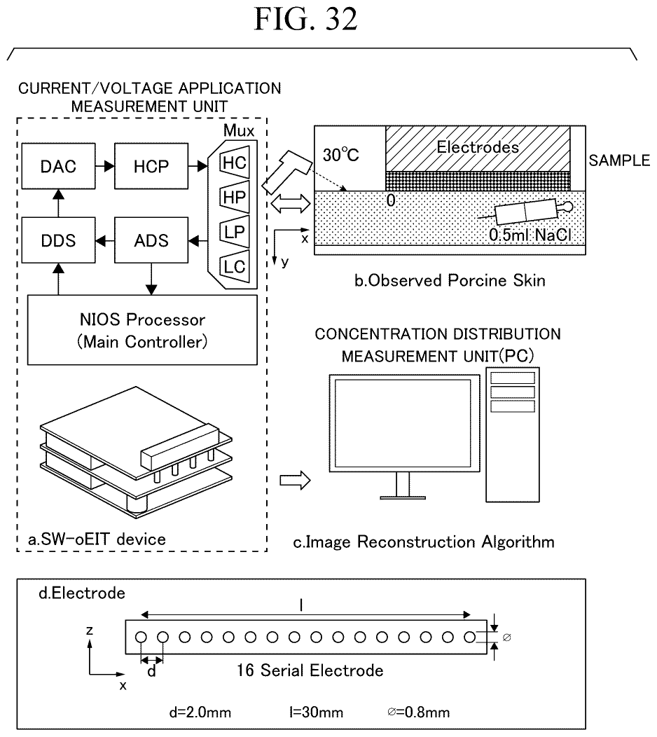

To experimentally demonstrate the third embodiment, FIG. 32 shows a configuration of the internal skin measurement apparatus used in Example 4. The internal skin measurement apparatus generates a square-wave signal and manages a measurement signal. The Hawland current supplies a constant current i to a wide range of frequencies. The pig skin was connected to 16 electrodes e1, e2, . . . , and e16 in one row with an electrode interval d=2 mm and an electrode diameter φ=0.8 mm. The switching of the electrodes was performed by a 16-channel analog multiplexer ADG1406. A dual simultaneous analog/digital converter LTC2145 was used for the high-speed data acquisition.

In Example 4, the four-terminal quasi-adjacent method from 16 electrodes was used. The measured voltage v_m was transmitted to the concentration distribution measurement unit 50 (computer).

In the acquisition process, to avoid oversampling and to keep the number N of potential difference samples constant at 2048, the sampling time Δt was controlled. By keeping N at a constant value, the speed of the FFT processing can be maintained.

To vary the sodium ion concentration in the dermis layer, the aqueous sodium chloride solution was injected into the dermis layer. The pig skin was cut into 10×40 mm, and 0.5 ml of the aqueous sodium chloride solution was directly injected into the dermis layer. The sodium chloride concentration was set to 5, 10, 15, 20, 25, 30, 35, 40, 45, and 50 [mM]. There were five skin samples for each condition, and each sample was measured five times to ensure the reproducibility and accuracy of the measurement.

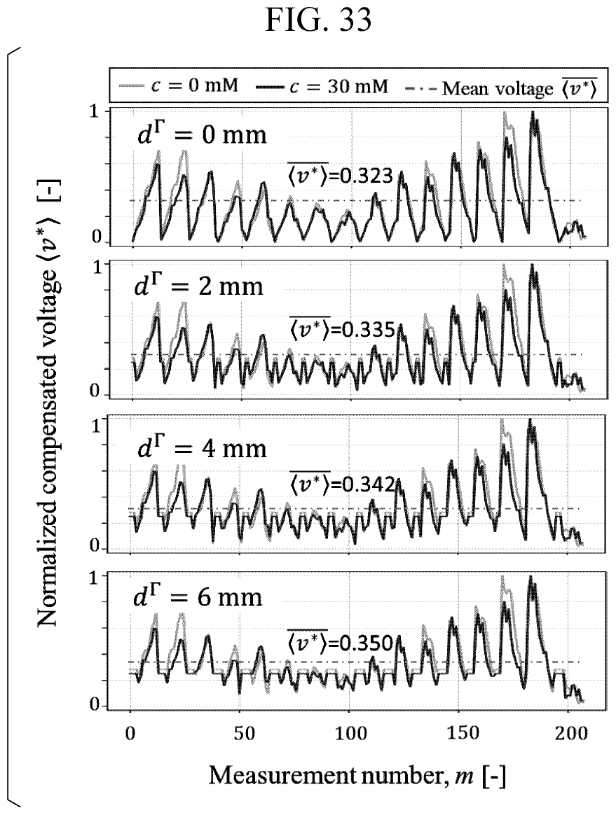

FIG. 33 shows a pattern change in the normalized compensation voltage <v*> in a case where the threshold value d^{Γ} was changed. It was confirmed that there was a significant change in the pattern cut off by the threshold value d^{Γ}. The larger the threshold value d^{Γ} was, the higher the normalized compensation voltage <v*> was.

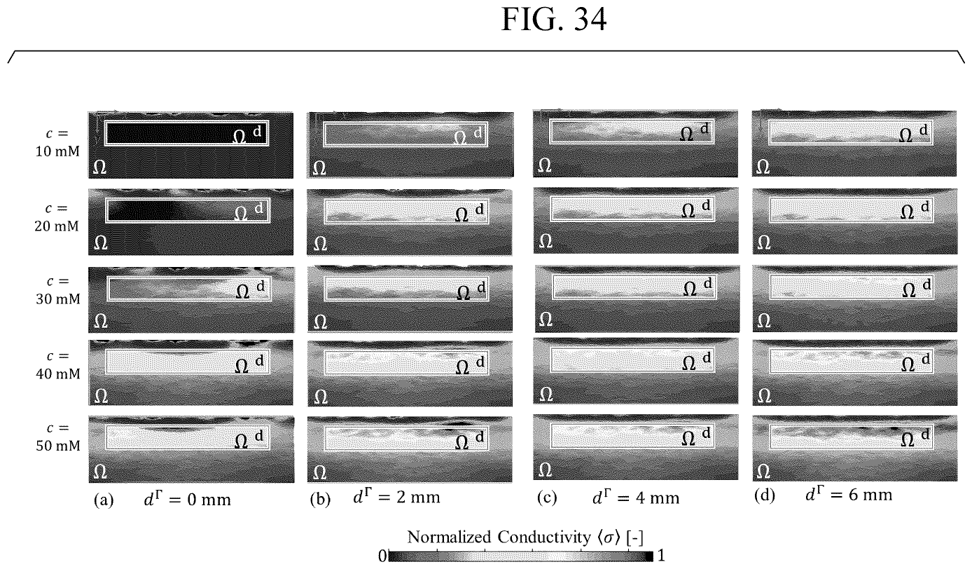

FIG. 34 shows an experimental result of a reconstructed image of the sodium ion concentration distribution in a case where the threshold value dΓ and the sodium chloride aqueous solution concentration were changed. In a case of the threshold value dΓ=0 mm, artifacts appeared in the stratum corneum layer and epidermis regions in the reconstructed image of the sodium ion concentration distribution c. The c of the epidermis layer was still dominant. On the other hand, in a case of dΓ=2 mm, 4 mm, and 6 mm, the image of the c distribution was more clearly emphasized in the region of interest ROI (region surrounded by a quadrangle in each figure) of the dermis layer, and the accuracy of quantification was improved.

### Example 5

To experimentally demonstrate the effect of the combination of the third embodiment and the other form (here, the first embodiment), the predetermined processing was performed in accordance with the threshold value based on the inter-electrode distance, and the potential difference or the current including the information on the concentration distribution of the target component (electrolyte, blood protein, or the like) in the specific skin layer of interest was amplified.

Specifically, the sodium ion concentration of the dermis layer was extracted by the Skip method.

In the Skip method, a planar sensor array having 16 electrodes and a quasi-adjacent injection pattern for extracting the sodium ion concentration of the dermis layer were adopted.

For each impedance measurement, four electrodes (high current (Hc), high potential (Hp), low potential (Lp), and low current (Lc)) are used.

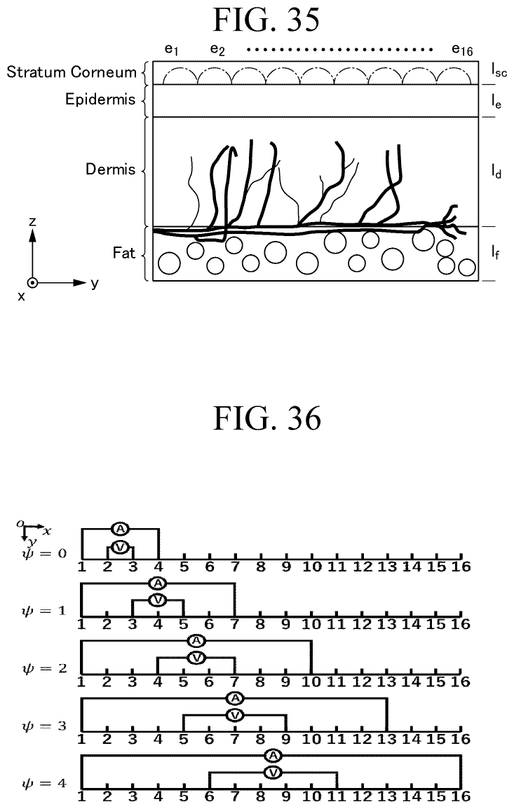

The quasi-adjacent method in the related art is regarded as Skip=0. Skip=1 represents a one-electrode skip between each adjacent working electrode, and Skip=2, Skip=3, and Skip=4 mean skips of two, three, and four electrodes between each adjacent working electrode (FIG. 36).

The threshold value method was used to determine the skip distance that was optimal for the sodium ion concentration extraction by varying the Skip (from Skip=0 to Skip=4).

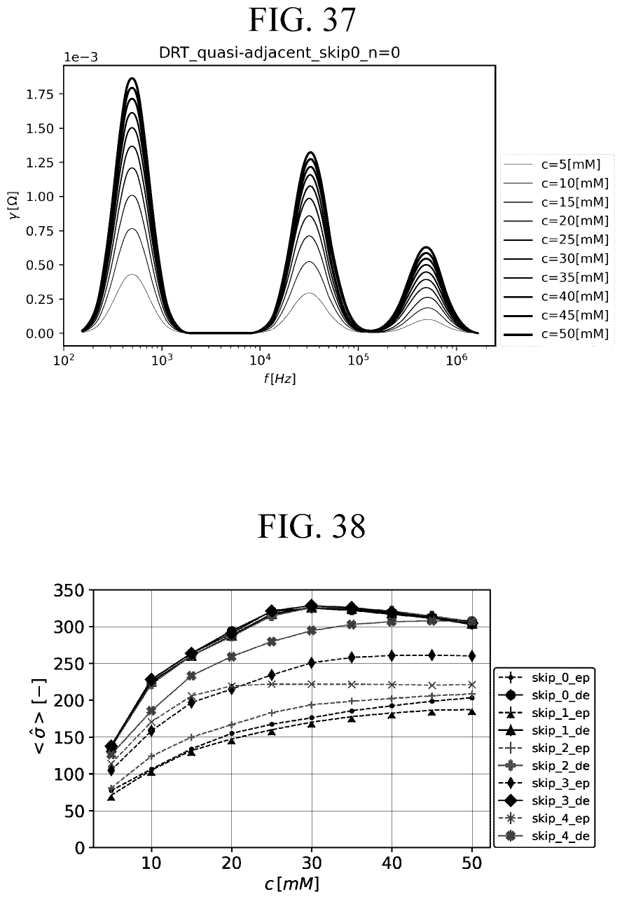

The DRT was used to determine the frequency that was optimal for the EIT image reconstruction (FIG. 37).

The performance of each skip variation was evaluated based on the conductivity change detected in both the dermis layer and the epidermis layer.

As a result of the present experiment, it was shown that all the variations of the Skip effectively detected the change in the sodium ion concentration of the dermis layer (FIG. 38).

However, as shown by the dashed line, the image artifact appearing as the conductivity change of the epidermis layer was observed in all the skip variations.

The variation of Skip=1 (solid line and dashed line connecting the triangular symbols) exhibited excellent performance in that the change in the conductivity of the epidermis layer is the least noticeable with respect to changes in the sodium ion concentration, while accurately capturing changes in the sodium ion concentration in the dermis layer and minimizing image artifacts.

The integration of the Skip method based on the threshold value method and the DRT provides a promising approach to extract the sodium ion concentration of the dermis layer while improving the image quality and the depth resolution. By systematically optimizing the Skip method and utilizing the DRT, excellent performance was achieved in the extraction of the sodium ion concentration of the dermis layer while minimizing the image artifact.

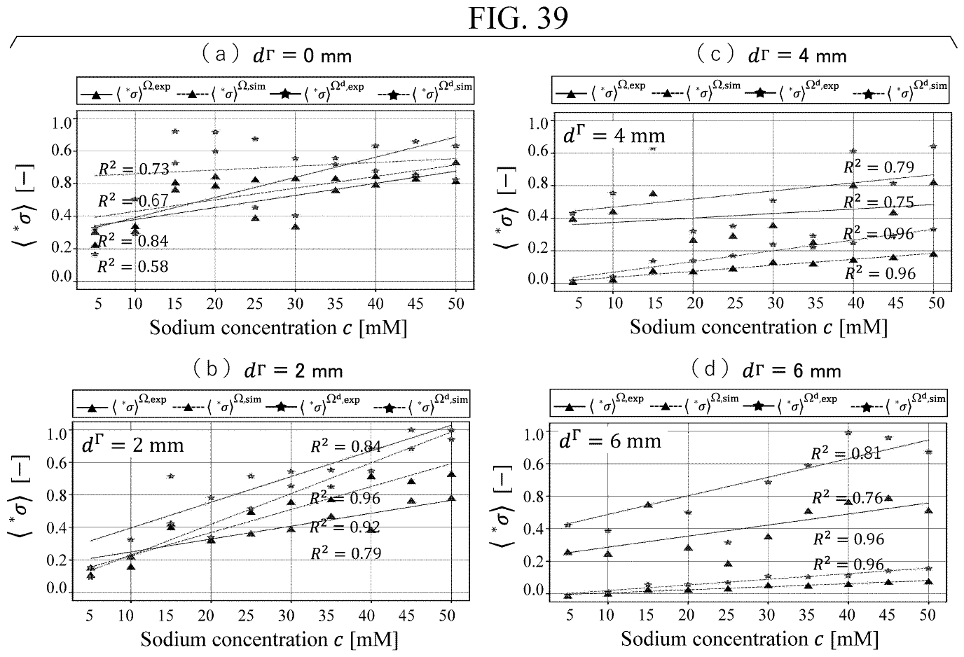

FIG. 39 shows a comparison of spatially averaged conductivity distribution <σ*> by the electromagnetic simulation and the experiment. FIG. 39A is a result for dΓ=0 mm, FIG. 39B is a result for dΓ=2 mm, FIG. 39C is a result for dΓ=4 mm, and FIG. 39D is a result for dΓ=8 mm. A horizontal axis of each figure is the sodium chloride aqueous solution concentration (mM), and a vertical axis is the spatially averaged conductivity distribution <σ*>. Ω represents <σ*> of the skin region, and Ωd represents <σ*> of the ROI region. The subscript exp indicates an experimental value, and the subscript sim indicates an electromagnetic simulation value.

Overall, <σ*> gradually increased as c increased in the electromagnetic simulation and the experiment. It was found that the coefficient of determination R² below the ROI region Ωd was higher than that of the skin region Ω, and the VDT could further reduce the image artifact of the stratum corneum layer and emphasize the region of interest (ROI region) of the dermis layer Ωd.

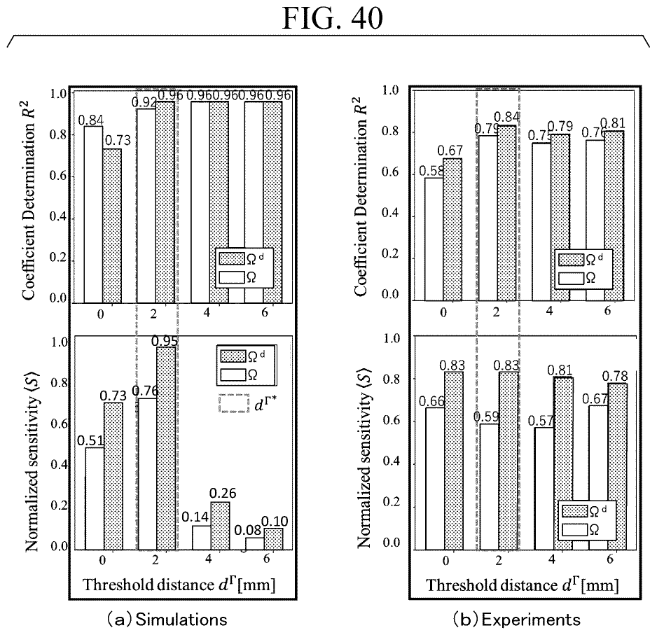

FIG. 40 shows an evaluation result of the threshold value dΓ by the electromagnetic simulation and the experiment. The upper figure of FIG. 40A shows a relationship between the coefficient of determination of the electromagnetic simulation and the threshold value, and the lower figure of FIG. 40A shows a relationship between the normalized sensitivity S of the electromagnetic simulation and the threshold value, and the upper figure of FIG. 40B shows a relationship between the coefficient of determination obtained from the experiment and the threshold value, and the lower figure of FIG. 40B shows a relationship between the normalized sensitivity S obtained from the experiment and the threshold value. From these results, it was found that the optimal threshold value (threshold value distance) was 2 mm.

### INDUSTRIAL APPLICABILITY

It is possible to provide an internal skin measurement apparatus that can separate each layer of the specific skin layer and understand the internal state of the skin based on the measured impedance.

### REFERENCE SIGNS LIST

10 Current/voltage injection measurement unit
20 Sensor
21 Electrode
30 Control unit
50 Concentration distribution measurement unit
51 Specific skin layer separation unit
52 Distribution function estimation unit
53 Calibration curve comparison unit, Output unit
60, 60A Concentration calculation unit
100 Internal skin measurement apparatus

1. An internal skin measurement apparatus comprising:
a current/voltage injection measurement unit that has a sensor including a plurality of electrodes configured to be arranged on a skin at intervals from each other; and
a concentration distribution measurement unit configured to measure at least one of an electrolyte concentration distribution or a blood protein concentration distribution of a specific layer inside the skin,
wherein the concentration distribution measurement unit includes a specific skin layer separation unit configured to calculate at least one feature as described below, the feature including information on the specific layer inside the skin and being obtained based on variation quantities of a current, a potential difference, and/or a phase measured by the current/voltage injection measurement unit,
(1) a feature based on a variation quantity between an impedance Z1(f) between distant electrodes through which an electric field penetrates a specific skin layer and an impedance Z2(f) between close electrodes through which the electric field does not penetrate the specific skin layer but penetrates only an upper layer of the specific skin layer,
(2) a feature based on variation quantities of a potential difference and a phase between the distant electrodes and between the close electrodes in a case where a square-wave current is applied, or a feature based on variation quantities of a current and a phase between the distant electrodes and between the close electrodes in a case where a square-wave voltage is applied, or
(3) a feature obtained by performing predetermined processing in accordance with a threshold value based on an inter-electrode distance, amplifying a potential difference or a current including information on a concentration distribution of a target component in the specific skin layer of interest, and reducing a potential difference or a current including information on the specific skin layer of no interest, and
the concentration distribution measurement unit includes a concentration calculation unit configured to calculate, using the feature, at least one of a conductivity, a permittivity, or a phase, which is correlated with an electrolyte concentration and a blood protein concentration in the specific layer.

2. The internal skin measurement apparatus according to claim 1,
wherein the concentration calculation unit includes a calibration curve comparison unit configured to calculate a concentration of an electrolyte or a blood protein based on
(a) a calibration curve of an electrolyte concentration with respect to a distribution function based on a relaxation time, which has been created prior to the calculation of the concentration of the electrolyte or the blood protein, and a distribution function based on the relaxation time, which is estimated from the feature, or
(b) a calibration curve of an electrolyte concentration with respect to a power spectral density drop, which has been created prior to the calculation of the concentration of the electrolyte or the blood protein, and a power spectral density drop, which is estimated from the feature.

3. The internal skin measurement apparatus according to claim 2,
wherein the concentration calculation unit includes the calibration curve comparison unit configured to calculate the concentration of the electrolyte or the blood protein based on a calibration curve of the electrolyte concentration with respect to the distribution function based on the relaxation time, which has been created prior to the calculation of the concentration of the electrolyte or the blood protein, and the distribution function based on the relaxation time, which is estimated from the feature.

4. The internal skin measurement apparatus according to claim 2,
wherein the concentration calculation unit includes a calibration curve comparison unit configured to calculate the concentration of the electrolyte or the blood protein based on the calibration curve of the electrolyte concentration with respect to the power spectral density drop, which has been created prior to the calculation of the concentration of the electrolyte or the blood protein, and the power spectral density drop, which is estimated from the feature.

5. The internal skin measurement apparatus according to claim 1,
wherein the concentration distribution measurement unit includes
a specific skin layer separation unit configured to perform the predetermined processing on the current or the potential difference measured by the current/voltage injection measurement unit in accordance with the threshold value based on the inter-electrode distance; and
an electrical property distribution calculation unit configured to calculate at least one of a conductivity distribution, a permittivity distribution, or a phase distribution based on the current or the potential difference after the predetermined processing.

6. A measurement method of an electrolyte concentration distribution and a blood protein concentration distribution in a specific skin layer, the measurement method comprising:
calculating at least one feature as described below, the feature including information on the specific layer inside the skin and being obtained based on variation quantities of a current, a potential difference, and/or a phase measured by the current/voltage injection measurement unit that has a sensor including a plurality of electrodes configured to be arranged on a skin at intervals from each other,
(1) a feature based on a variation quantity between an impedance Z1(f) between distant electrodes through which an electric field penetrates a specific skin layer and an impedance Z2(f) between close electrodes through which the electric field does not penetrate the specific skin layer but penetrates only an upper layer of the specific skin layer,
(2) a feature based on variation quantities of a potential difference and a phase between the distant electrodes and between the close electrodes in a case where a square-wave current is applied, or a feature based on variation quantities of a current and a phase between the distant electrodes and between the close electrodes in a case where a square-wave voltage is applied, or
(3) a feature obtained by performing predetermined processing in accordance with a threshold value based on an inter-electrode distance, amplifying a potential difference or a current including information on a concentration distribution of a target component in the specific skin layer of interest, and reducing a potential difference or a current including information on the specific skin layer of no interest; and
using a result obtained by calculating, using the feature, at least one of a conductivity, a permittivity, or a phase, which is correlated with an electrolyte concentration and a blood protein concentration in the specific layer.

7. The measurement method according to claim 6,
wherein the calculation using the feature is a calculation of a concentration of an electrolyte or a blood protein based on
(a) a calibration curve of a distribution function based on the electrolyte concentration and a relaxation time, which has been created prior to the calculation of the concentration of the electrolyte or the blood protein, and a distribution function based on the relaxation time, which is estimated from the feature, or
(b) a calibration curve of an electrolyte concentration and a power spectral density drop, which has been created prior to the calculation of the concentration of the electrolyte or the blood protein, and a power spectral density drop, which is estimated from the feature.
